(19) United States
(12) Patent Application Publication
(10) Pub. No.: US 2024/0165224 A1
(43) Pub. Date: May 23, 2024

(54) IMMUNOGENIC COMPOSITIONS

(71) Applicants: GLAXOSMITHKLINE BIOLOGICALS SA, Rixensart (BE); UNITED STATES OF AMERICA, as represented by THE SECRETARY, DEPARTMENT OF HEALTH AND HUMAN SERVICES, Bethesda, MD (US)

(72) Inventors: Ventzislav Bojidarov VASSILEV, Rixensart (BE); Corey MALLETT, Rockville, MD (US); Ronan ROUXEL, Rixensart (BE); Normand BLAIS, Rixensart (BE); Masaru KANEKIYO, Bethesda, MD (US); Barney S. GRAHAM, Bethesda, MD (US)

(73) Assignees: GLAXOSMITHKLINE BIOLOGICALS SA, Rixensart (BE); UNITED STATES OF AMERICA, as represented by THE SECRETARY, DEPARTMENT OF HEALTH AND HUMAN SERVICES, Bethesda, MD (US)

(21) Appl. No.: 18/283,516

(22) PCT Filed: Mar. 25, 2022
(86) PCT No.: PCT/EP2022/057945

§ 371 Date: Sep. 22, 2023

Related U.S. Application Data
(60) Provisional application No. 63/166,556, filed on Mar. 26, 2021.

(51) Int. Cl. A61K 39/39; A61K 39/00; A61K 39/145; A61P 31/16; A61P 37/04

(52) U.S. Cl. CPC A61K 39/39; A61K 39/145; A61P 31/16; A61P 37/04; A61K 2039/55566; A61K 2039/6031

(57) ABSTRACT

The present invention relates to influenza virus immunisation using at least one influenza HA stem polypeptide in conjunction with squalene emulsion adjuvants, and to related aspects.

### TECHNICAL FIELD

The present invention relates to influenza immunisation using an isolated hemagglutinin (HA) stem polypeptide in conjunction with squalene emulsion adjuvants, and to related aspects.

### BACKGROUND

Influenza viruses have a significant impact on global public health, causing millions of cases of severe illness each year, thousands of deaths, and considerable economic losses. Current trivalent influenza vaccines elicit antibody responses to the vaccine strains and closely related isolates, but rarely extend to more diverged strains within a subtype or to other subtypes. In addition, selection of the appropriate vaccine strains presents many challenges and frequently results in sub-optimal protection.

Protective immune responses induced by vaccination against influenza viruses are primarily directed to the viral HA protein, which is a glycoprotein on the surface of the virus responsible for interaction of the virus with host cell receptors. HA proteins on the virus surface are trimers of HA protein monomers that are enzymatically cleaved to yield amino-terminal HA1 and carboxy-terminal HA2 polypeptides. The globular head consists exclusively of the major portion of the HA1 polypeptide, whereas the stem that anchors the HA protein into the viral lipid envelope is comprised of HA2 and part of HA1. The globular head of a HA protein includes two domains: the receptor binding domain (RBD), a domain that includes the sialic acid-binding site, and the vestigial esterase domain, a smaller region just below the RBD. The globular head includes several antigenic sites that include immunodominant epitopes.

Antibodies against influenza often target variable antigenic sites in the globular head of HA and thus, neutralize only antigenically closely related viruses. The variability of the HA head is due to the constant antigenic drift of influenza viruses and is responsible for seasonal endemics of influenza. In contrast, the HA stem is highly conserved and experiences little antigenic drift.

An entirely new class of broadly neutralizing antibodies against influenza viruses has been isolated that recognize the highly conserved HA stem (Corti, 2011). Unlike strain-specific antibodies, antibodies in this new class are capable of neutralizing multiple antigenically distinct viruses. However, robustly eliciting these antibodies in subjects by vaccination with the HA stem, lacking the head domain, has been difficult (Steel, 2010). Removal of the immunodominant head region of HA (which contains competing epitopes) and stabilization of the resulting stem region through genetic manipulation is one potential way to improve the elicitation of these broadly neutralizing stem antibodies.

Advances in biotechnology in past decades have allowed engineering of biological materials to be exploited for the generation of novel vaccine platforms. Ferritin, an iron storage protein found in almost all living organisms, is an example which has been extensively studied and engineered for a number of potential biochemical/biomedical purposes. The use of ferritin self-assembling nanoparticles to present stabilised stem trimers is described in Corbett, 2019.

Oil-in-water emulsion adjuvants containing squalene have featured in licensed pandemic and prepandemic influenza vaccines. ‘AS03’ (WO2006/100109; Gargon, 2012; Cohet, 2019) includes squalene, alpha-tocopherol and polysorbate 80. An adult human dose of AS03_{A} contains 10.69 mg squalene, 11.86 mg alpha-tocopherol and 4.86 mg polysorbate 80 (Fox, 2009; Morel, 2011). Certain reduced does of AS03 have also been described (WO2008/043774), including AS03_{B} (½ dose), AS03c (¼ dose) and AS03_{D} (⅛ dose) (Carmona Martinez, 2014). AS03 and MF59 (a submicron oil-in-water emulsion of squalene, polysorbate 80 and sorbitan trioleate) adjuvants have been shown to augment the immune responses to 2 doses of an inactivated H7N9 influenza vaccine, with the AS03-adjuvanted formulations inducing the highest titers (Jackson, 2015).

Stable emulsions (SE) have also been described which contain squalene, phospholipid, poloxamer 188 (Pluronic F68) and glycerol in ammonium phosphate buffer (Carter, 2016). The SE have sometimes been described as containing low levels of alpha-tocopherol as an antioxidant (Sun, 2016).

There remains a need for an influenza vaccine that provides a broad and robust immune response against influenza virus. There particularly remains a need for an influenza vaccine that protects individuals from heterologous strains of influenza virus (i.e. a ‘universal’ vaccine’), including evolving seasonal and pandemic influenza virus strains of the future.

### SUMMARY OF THE INVENTION

It has been found that squalene emulsion adjuvants enhance the immunogenicity of the influenza HA stem region.

In particular, or in addition, influenza HA stem polypeptides adjuvanted with squalene emulsion adjuvants according to the invention, induce a homologous, a heterologous and/or a heterosubtypic cross-reactive immunogenic response against influenza virus, preferably against Influenza A virus, more preferably against Influenza A virus subtypes of Group 1 and/or Group 2.

The invention therefore provides a method of eliciting an immune response in a subject, the method comprising administering to the subject (i) at least one isolated influenza HA stem polypeptide and (ii) a squalene emulsion adjuvant.

Also provided is a method of adjuvanting the immune response of a subject to at least one isolated influenza HA stem polypeptide, the method comprising administering to the subject a squalene emulsion adjuvant.

Also provided is a squalene emulsion adjuvant for use in eliciting an immune response in a subject by administration with at least one isolated influenza HA stem polypeptide.

Also provided is at least one isolated influenza HA stem polypeptide, for use in eliciting an immune response in a subject by administration with a squalene emulsion adjuvant.

Also provided is the use of a squalene emulsion adjuvant in the manufacture of a medicament for use in eliciting an immune response in a subject by administration with at least one isolated influenza HA stem polypeptide.

Also provided is the use of at least one isolated influenza HA stem polypeptide in the manufacture of a medicament for use in eliciting an immune response in a subject by administration with a squalene emulsion adjuvant.

Also provided is a kit comprising:
(i) a first container comprising at least one isolated influenza HA stem polypeptide; and
(ii) a second container comprising a squalene emulsion adjuvant.

Also provided is an immunogenic composition comprising: (i) at least one isolated influenza HA stem polypeptide, and (ii) a squalene emulsion adjuvant.

Also provided is the use of (i) at least one isolated influenza HA stem polypeptide, and (ii) a squalene emulsion adjuvant, in the manufacture of a medicament.

Further embodiments of the invention are provided in the text below.

### BRIEF DESCRIPTION OF THE SEQUENCES

SEQ ID NO: 1: Polypeptide sequence of stabilised HA stem from A/New Caledonia/20/1999 (H1N1)
SEQ ID NO: 2: Polypeptide sequence of stabilised HA stem from A/Michigan/45/2015 (H1N1)
SEQ ID NO: 3: Polypeptide sequence of stabilised HA stem from A/Finland/486/2004 (H3N2)
SEQ ID NO: 4: Polypeptide sequence of stabilised HA stem from A/Jiangxi/IPB13/2013 (H10N8), also referred to as A/Jiangxi-Donghu/346/2013
SEQ ID NO: 5: Polypeptide sequence of H. pylori ferritin
SEQ ID NO: 6: Polypeptide sequence of H1ssF_pylori (signal peptide-stabilised HA stem from A/New Caledonia/20/1999 (H1N1)-SGG-H. pylori ferritin)
SEQ ID NO: 7: Polypeptide sequence of H1ssF_pylori (signal peptide-stabilised HA stem from A/Michigan/45/2015 (H1N1)-SGG-H. pylori ferritin)
SEQ ID NO: 8: Polypeptide sequence of H3ssF_pylori (signal peptide-stabilised HA stem from A/Finland/486/2004 (H3N2)-SGG-H. pylori ferritin)
SEQ ID NO: 9: Polypeptide sequence of H10ssF_pylori (signal peptide-stabilised HA stem from A/Jiangxi/IPB13/2013 (H10N8)-SGG-H. pylori ferritin)
SEQ ID NO: 10: Polypeptide sequence of insect ferritin, heavy chain from Trichoplusia ni
SEQ ID NO: 11: Polypeptide sequence of insect ferritin, light chain from Trichoplusia ni
SEQ ID NO: 12: GGSGG linker sequence
SEQ ID NO: 13: Polypeptide sequence of insect ferritin construct (iFH-F2A-iFL-6R; single polypeptide, self-cleaving; insect ferritin heavy chain-self-cleaving sequence-insect ferritin light chain)
SEQ ID NO: 14: Polypeptide sequence of H1ss_iH-F2A-H3ss_iL-6R (single polypeptide, self-cleaving; signal peptide-stabilised HA stem from A/Michigan/45/2015 (H1N1)-GGSGG-insect ferritin heavy chain-self-cleaving sequence-signal peptide-stabilised HA stem from A/Finland/486/2004 (H3N2)-insect ferritin light chain)
SEQ ID NO: 15: Polypeptide sequence of H1Mich15ss_iH-F2A-H10ss_iL-6R (single polypeptide, self-cleaving; signal peptide-stabilised HA stem from A/Michigan/45/2015 (H1N1)-GGSGG-insect ferritin heavy chain-self-cleaving sequence-signal peptide-stabilised HA stem from A/Jiangxi/IPB13/2013 (H10N8)-insect ferritin light chain)
SEQ ID NO: 16: Polypeptide sequence of H1NC99ss_iH-F2A-H10ss_iL-6R (single polypeptide, self-cleaving; signal peptide-stabilised HA stem from A/New Caledonia/20/1999 (H1N1)-GGSGG-insect ferritin heavy chain-self-cleaving sequence-signal peptide-stabilised HA stem from A/Jiangxi/IPB13/2013 (H10N8)-insect ferritin light chain)

### DESCRIPTION OF THE FIGURES

FIG. 1: Study A: Anti-H1 stem IgG antibody titers by ELISA at 14 days post dose 2

FIG. 2: Study B: Anti-H1 stem IgG antibody titers by ELISA at 14 days post dose 2

FIG. 3: Study C: Anti-H1 stem IgG antibody titers by ELISA at 14 days post dose 2

FIG. 4: Study A: Anti-H1/NC/99 IgG antibody titers by ELISA at 14 days post dose 2

FIG. 5: Study B: Anti-H1/NC/99 IgG antibody titers by ELISA at 14 days post dose 2

FIG. 6: Study A: Anti-H1/Mich/15 IgG antibody titers by ELISA at 14 days post dose 2

FIG. 7: Study B: Anti-H1/Mich/15 IgG antibody titers by ELISA at 14 days post dose 2

FIG. 8: Study C: Anti-H1/Mich/15 IgG antibody titers by ELISA at 14 days post dose 2

FIG. 9: Study A: Anti-H2/Neth/99 IgG antibody titers by ELISA at 14 days post dose 2

FIG. 10: Study B: Anti-H2/Neth/99 IgG antibody titers by ELISA at 14 days post dose 2

FIG. 11: Study C: Anti-H2/Neth/99 IgG antibody titers by ELISA at 14 days post dose 2

FIG. 12: Study A: Anti-H9 IgG antibody titers by ELISA at 14 days post dose 2

FIG. 13: Study B: Anti-H9 IgG antibody titers by ELISA at 14 days post dose 2

FIG. 14: Study C: Anti-H9 IgG antibody titers by ELISA at 14 days post dose 2

FIG. 15: Study A: Anti-H18 IgG antibody titers by ELISA at 14 days post dose 2

FIG. 16: Study B: Anti-H18 IgG antibody titers by ELISA at 14 days post dose 2

FIG. 17: Study C: Anti-H18 IgG antibody titers by ELISA at 14 days post dose 2

FIG. 18: Study B: Anti-H3 IgG antibody titers by ELISA at 14 days post dose 2

FIG. 19: Study B: Anti-H7 IgG antibody titers by ELISA at 14 days post dose 2

FIG. 20: Study B: Anti-H10 IgG antibody titers by ELISA at 14 days post dose 2

FIG. 21: Study C: Anti-H3 IgG antibody titers by ELISA at 14 days post dose 2

FIG. 22: Study C: Anti-H7 IgG antibody titers by ELISA at 14 days post dose 2

FIG. 23: Study C: Anti-H10 IgG antibody titers by ELISA at 14 days post dose 2

FIG. 24: Study A: Percentage of stem H1/Mich/2015 specific CD4+ T cell at 14 days post dose 2

FIG. 25: Study B: Percentage of stem specific CD4+ T cell at 14 days post dose 2

FIG. 26: Study C: Percentage of stem specific CD4+ T cell at 14 days post dose 2

FIG. 27: Study A: Percentage of stem H1/Mich/2015 specific CD8+ T cell at 14 days post dose 2

FIG. 28: Study B: Percentage of stem specific CD8+ T cell at 14 days post dose 2

FIG. 29: Study C: Percentage of stem specific CD8+ T cell at 14 days post dose 2

FIG. 30: Study B: Percentage of stem H10/Jiangxi-Donghu specific CD4+ T cell at 14 days post dose 2

FIG. 31: Study B: Percentage of stem H10/Jiangxi-Donghu specific CD8+ T cell at 14 days post dose 2

FIG. 32: Study C: Percentage of stem H10/Jiangxi-Donghu specific CD4+ T cell at 14 days post dose 2

FIG. 33: Study C: Percentage of stem H10/Jiangxi-Donghu specific CD8+ T cell at 14 days post dose 2

FIG. 34: Microneutralization titers against H1/Mich/15, H1/NC/99 and H5/Vn/04 at 14 days post dose 2

FIG. 35: Schematic of HA stem-H. pylori ferritin inserts

### DETAILED DESCRIPTION OF THE INVENTION

### Isolated Influenza HA Stem Polypeptide

Influenza hemagglutinin (HA) is the major surface antigen of the virion and the primary target of virus neutralizing antibodies. HA is a homotrimeric surface glycoprotein, with each monomer consisting of two disulfide-linked subunits (HA1, HA2), resulting from the proteolytic cleavage products of a single HA precursor protein. The HA1 chain forms a membrane-distal globular head and a part of the membrane-proximal stem (or ‘stalk’) region. The HA2 chain represents the major component of the stem region. The head of HA mediates receptor binding while the membrane-anchored stem is the main part of membrane fusion machinery. The invention disclosed herein relates to the influenza HA stem region when isolated from the influenza HA head region. The invention disclosed herein does not relate to the influenza HA stem region when comprised within the whole influenza HA polypeptide.

An ‘isolated influenza HA stem polypeptide’ as used herein refers to a polypeptide comprising a full length influenza HA stem region or an immunogenic fragment or variant of an influenza HA stem region. In one embodiment the isolated influenza HA stem polypeptide is a polypeptide consisting of a full length influenza HA stem region or an immunogenic fragment or variant of an influenza HA stem region.

In one embodiment the isolated influenza HA stem polypeptide is desirably 400 residues or fewer in length, especially 300 residues or fewer, in particular 250 residues or fewer, such as 220 residues or fewer. In one embodiment the isolated influenza HA stem polypeptide is desirably 130 residues or more in length, especially 160 residues or more, in particular 180 residues or more, such as 190 residues or more. In one embodiment the isolated influenza HA stem polypeptide is desirably 130 to 400 residues in length, especially 160 to 300, in particular 180 to 250, such as 190 to 220.

In some embodiments, the influenza HA stem polypeptide comprises or consists of an amino acid sequence having at least 90%, 95%, 98% or 99% identity to the amino acid sequence set forth in any one of SEQ ID NO:1, SEQ ID NO: 2, SEQ ID NO: 3 or SEQ ID NO: 4.

In some preferred embodiments, the influenza HA stem polypeptide comprises or consists of the amino acid sequence set forth in any one of SEQ ID NO: 1, SEQ ID NO: 2, SEQ ID NO: 3 or SEQ ID NO: 4.

Suitably the isolated influenza HA stem polypeptide is derived from type A or B influenza virus. More suitably the isolated influenza HA stem polypeptide is derived from type A influenza virus.

In one embodiment the influenza HA stem polypeptide is derived from influenza A, such as influenza A Group 1, or alternatively influenza A HA subtype H1, H2, H3, H7 or H10, more suitably H1 or H10, more suitably H1. In an alternative embodiment the influenza HA stem polypeptide is derived from influenza B. In one embodiment the isolated influenza HA stem polypeptide is not derived from influenza A HA subtype H8, such as not derived from influenza A HA H9 clade (H8, H9 and H12).

In some embodiments, the influenza HA stem polypeptide is derived from influenza A Group1, suitably subtypes H1, H2, H5, H6, H8, H9, H11, H12, H13, H16, H17 or H18, preferably H1.

In some particular embodiments, the influenza HA stem polypeptide comprises or consists of an amino acid sequence having at least 90%, 95%, 98% or 99% identity to the amino acid sequence set forth in any one of SEQ ID NO:1 or SEQ ID NO: 2, preferably SEQ ID NO: 2.

In some preferred embodiments, the influenza HA stem polypeptide comprises or consists of the amino acid sequence set forth in any one of SEQ ID NO:1 or SEQ ID NO: 2, preferably SEQ ID NO: 2.

In some embodiments, the influenza HA stem polypeptide is derived from influenza A Group 2, suitably subtypes H3, H4, H7, H10, H14 and H15, preferably H3 or H10, more preferably H10.

In some embodiments, the influenza HA stem polypeptide comprises or consists of an amino acid sequence having at least 90%, 95%, 98% or 99% identity to the amino acid sequence set forth in any one of SEQ ID NO: 3 or SEQ ID NO: 4.

In some preferred embodiments, the influenza HA stem polypeptide comprises or consists of the amino acid sequence set forth in any one of SEQ ID NO: 3 or SEQ ID NO: 4.

In further embodiments, two or more different influenza HA stem polypeptides are present, such as a first and a second influenza HA stem polypeptide which are derived from different influenza strains and/or subtypes and/or groups, such as one from group A1 and one from group A2 for example one from an H1 strain and one from an H3 or an H10 strain. In particular embodiments, the two or more influenza HA stem polypeptides each comprise or consist of an amino acid sequence having at least 90%, 95%, 98% or 99% identity to the amino acid sequence set forth in any one of SEQ ID NO:1, SEQ ID NO: 2, SEQ ID NO: 3 or SEQ ID NO: 4, such as a combination of SEQ ID NO: 1 and 3, or 1 and 4, or 2 and 3, or 2 and 4. More particularly, the two or more influenza HA stem polypeptides each comprise or consist of an amino acid sequence set forth in any one of SEQ ID NO: 1, SEQ ID NO: 2, SEQ ID NO: 3 or SEQ ID NO: 4, such as a combination of SEQ ID NO: 1 and 3, or 1 and 4, or 2 and 3, or 2 and 4.

The influenza HA stem polypeptide used herein is ‘isolated’. By ‘isolated’, it is meant that the influenza HA stem polypeptide does not additionally comprise an influenza HA head region. Accordingly, the isolated influenza HA stem polypeptide does not comprise an influenza HA head region, and more suitably the isolated influenza HA stem polypeptide does not comprise any additional regions from influenza HA. The isolated influenza HA stem polypeptide is not a full length influenza HA protein.

The isolated influenza HA stem polypeptide may be comprised within a construct which comprises further polypeptide sequences. The further polypeptide sequences may include, for example, one or more signal peptides and/or one or more linkers. In some embodiments, the signal peptide is not present in the final construct of the compositions or methods or uses herein.

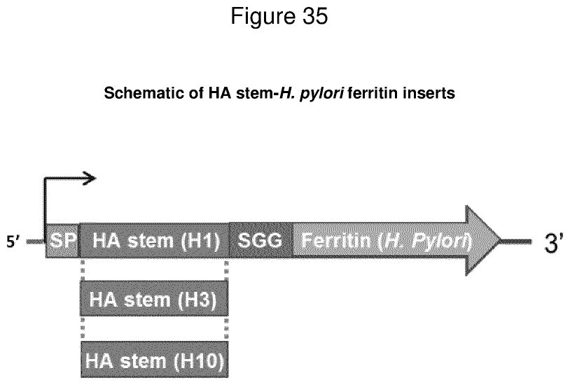

The isolated influenza HA stem polypeptide used in some examples (e.g. Study A and Study B) is comprised within a construct which also includes a signal peptide (SP), stabilised HA stem, a serine-glycine-glycine (SGG) linker, and H. pylori ferritin. The construct has the format: signal peptide (SP)-stabilised HA stem-SGG-H. pylori ferritin (FIG. 35). The polypeptide sequences of the specific constructs used in these examples are SEQ ID NO: 6 (signal peptide-stabilised HA stem from A/New Caledonia/20/1999 (H1N1)-SGG-H. pylori ferritin), SEQ ID NO: 7 (signal peptide-stabilised HA stem from A/Michigan/45/2015 (H1N1)-SGG-H. pylori ferritin), and SEQ ID NO: 9 (signal peptide-stabilised HA stem from A/Jiangxi/IPB13/2013 (H10N8)-SGG-H. pylori ferritin). A further analogous construct which comprises an alternative HA stem polypeptide has the polypeptide sequence given in SEQ ID NO: 8 (signal peptide-stabilised HA stem from A/Finland/486/2004 (H3N2)-SGG-H. pylori ferritin). In some embodiments the signal peptide is not present in the final construct. Accordingly, in one embodiment, the influenza stem polypeptide is comprised within a construct having a polypeptide sequence having 80% or greater, such as 90% or greater, such as 95% or greater, such as 98% or greater, such as 99% or greater sequence identity to any one of SEQ ID NO: 6-9, with or without the signal peptide, more suitably any one of SEQ ID NO: 6, 7 or 9, more suitably SEQ ID NO: 6 or 7. Suitably the construct comprises or consists of any one of SEQ ID NOs: 6-9, more suitably SEQ ID NO: 6, 7 or 9, more suitably SEQ ID NO: 6 or 7, with or without the signal peptide.

The influenza HA stem polypeptides used in some further examples (e.g. Study C) are comprised within a construct which comprises a signal peptide (SP), stabilised HA stem, a glycine-glycine-serine-glycine-glycine (GGSGG) [SEQ ID NO: 12] linker, a self-cleaving sequence and insect ferritin.

In some embodiments, the ferritin such as insect ferritin comprises two monomeric subunits, such as a light chain and a heavy chain. According to some further embodiments, each monomeric subunit may be linked to an influenza HA stem polypeptide derived from one or more strains and/or one or more subtypes, preferably from one or more influenza A Group 1 subtypes and/or one or more influenza A Group 2 subtypes. In particular embodiments, the insect ferritin comprises a heavy chain and a light chain amino acid sequence comprising or consisting of a sequence having at least 90%, 95%, 98% or 99% identity to the amino acid sequence set forth SEQ ID NO:10 and SEQ ID NO: 11. In particular embodiments, the insect ferritin heavy and light chains comprise or consist of the sequences set forth in SEQ ID NO: 10 and 11.

In some embodiments, each monomeric subunit may be linked to an influenza HA stem polypeptide derived from one influenza A subtype, e.g. from Group 1 or Group 2. In one embodiment, the light chain and the heavy chain are both linked to an influenza HA stem polypeptide derived from the same subtype, such as derived from H1 or H3 or H10 subtype, such as from H1 or H10. In certain embodiments, the light chain and the heavy chain are both linked to an influenza HA stem polypeptide derived from the same influenza A strain, i.e. the influenza HA stem polypeptides linked to the light chain and the heavy chain are identical. In particular embodiments, the light chain and the heavy chain are both linked to an influenza HA stem polypeptide derived from A/Michigan/45/2015 (H1N1), A/Finland/486/2004 (H3N2) or A/Jiangxi/IPB13/2013 (H10N8). Where the light chain and heavy chains are linked to influenza HA stem polypeptides derived from the same influenza strain, this may be referred to as homotypic display.

In further embodiments, each monomeric subunit is linked to influenza HA stem polypeptide derived from at least two different influenza A strains, which may be from the same or different subtypes. In further embodiments, each monomeric subunit is linked to influenza HA stem polypeptide derived from a different subtype, e.g. from Group 1 and Group 2. Preferably, the light chain and the heavy chain are linked to influenza HA stem polypeptides derived from different influenza A subtypes, more preferably the light chain is linked to an influenza HA stem polypeptide derived from influenza A Group 1, preferably from H1 subtype, and the heavy chain is linked to an influenza HA stem polypeptide derived from influenza A Group 2, preferably from H3 or H10 subtype, or the reverse. According to certain embodiments, the heavy chain is linked to an influenza HA stem polypeptide derived from A/Michigan/45/2015 (H1N1) and the light chain is linked to an influenza HA stem polypeptide derived from A/Finland/486/2004 (H3N2) or A/Jiangxi/IPB13/2013 (H10N8). Where the light chain and heavy chains are linked to influenza HA stem polypeptides derived from different influenza strains, such as from different influenza A subtypes, this may be referred to as heterotypic display.

A polypeptide sequence used in some of the examples (Study C) is comprised within a construct shown in SEQ ID NO: 15 (H1/H10 ssF insect—HA stem from A/Michigan/45/2015 (H1N1) and from A/Jiangxi/IPB13/2013 (H10N8)). SEQ ID NO: 15 is a single peptide with the signal peptide and F2A cleavage site, which are absent in the monomeric subunits present in the insect ferritin nanoparticle. Further analogous constructs which comprises alternative combinations of HA stem polypeptides have the polypeptide sequence given in SEQ ID NO: 14 (H1/H3 ssF insect—HA stem from A/Michigan/45/2015 (H1N1) and from A/Finland/486/2004 (H3N2)) and SEQ ID NO: 16 (H1/H10 ssF insect-HA stem from A/New Caledonia/20/1999 (H1N1)- and from A/Jiangxi/IPB13/2013 (H10N8)).

Accordingly, in some embodiments, the influenza HA stem polypeptides are derived from a construct having a polypeptide sequence having 80% or greater, such as 90% or greater, such as 95% or greater, such as 98% or greater, such as 99% or greater sequence identity to any one of SEQ ID NO: 14 or 15 or 16, more suitably SEQ ID NO: 15. Suitably the construct comprises or consists of any one of SEQ ID NOs: 14, 15 or 16, more suitably SEQ ID NO: 15.

Suitably the immune response elicited by the isolated influenza HA stem polypeptide produces antibodies to influenza virus. More suitably, the elicited immune response produces anti-stem region antibodies.

A Type of influenza virus refers to influenza Type A, influenza Type B or influenza type C. The designation of a virus as a specific Type relates to sequence difference in the respective M1 (matrix) protein, M2 (ion channel) protein or NP (nucleoprotein). Type A influenza viruses are further divided into Group 1 and Group 2. These Groups are further divided into subtypes, which refers to classification of a virus based on the sequence of its HA protein. Examples of current commonly recognized subtypes are H1, H2, H3, H4, H5, H6, H7, H8, H9, H10, H11, H12, H13, H14, H15, H16, H17 or H18. Group 1 influenza subtypes are H1, H2, H5, H6, H8, H9, H11, H12, H13, H16, H17 and H18. Group 2 influenza subtypes are H3, H4, H7, H10, H14 and H15. Finally, the term strain refers to viruses within a subtype that differ from one another in that they have small, genetic variations in their genome.

In some embodiments the elicited immune response produces anti-Group 1 influenza A stem region antibodies, preferably anti-H1, H2, H9 and/or H18 stem region antibodies. In some embodiments, the elicited immune response produces anti-Group 2 influenza A stem region antibodies, preferably anti-H3, H7 and/or H10, more preferably anti-H7 and/or H10 stem region antibodies. Suitably the elicited immune response produces both anti-Group 1, preferably anti-H1, H2, H9 and/or H18 stem region antibodies, and anti-Group 2, preferably anti-H3, H7 and/or H10, more preferably anti-H7 and/or H10 influenza A stem region antibodies, for example anti-H1 and anti-H3 antibodies.

In some embodiments the elicited immune response produces one or more of anti-H1, H2, H3, H7, H9, H10 or H18 stem region antibodies. More suitably the elicited immune response produces one or more of anti-H1, H2, H7, H9, H10 or H18 stem region antibodies.

Suitably the elicited immune response produces all of anti-H1, H2, H3, H7, H9, H10 and H18 stem region antibodies. More suitably the elicited immune response produces all of anti-H1, H2, H7, H9, H10 and H18 stem region antibodies.

In some embodiments, the elicited immune response is homologous (against the same strain), heterologous (against different strains which may be within a subtype) and/or heterosubtypic cross-reactive (against a different strain or strains within one or more different subtypes, e.g. from Group 1 and/or from Group 2 subtypes).

### Full Length Influenza HA Stem Region

In one embodiment the isolated influenza HA stem polypeptide is a polypeptide comprising a full length influenza HA stem region. Suitably the isolated influenza HA stem polypeptide is a polypeptide consisting of a full length influenza HA stem region.

The full length influenza HA stem region is desirably 400 residues or fewer in length, especially 300 residues or fewer, in particular 250 residues or fewer, such as 220 residues or fewer. The full length influenza HA stem region is desirably 130 residues or more in length, especially 160 residues or more, in particular 180 residues or more, such as 190 residues or more.

Suitably the full length influenza HA stem region comprises or more suitably consists of a polypeptide sequence selected from SEQ ID NOs: 1-4. More suitably the full length influenza HA stem region comprises or more suitably consists of SEQ ID NO: 1, 2 or 4. More suitably the full length influenza HA stem region comprises or more suitably consists of SEQ ID NO: 2 or 4, still more suitably comprises or more suitably consists of SEQ ID NO: 2.

The isolated influenza HA stem polypeptide is also referred to herein as an ‘antigen’ or an ‘influenza stem polypeptide’.

Further suitable full length influenza HA stem regions are those disclosed in WO2013/044203, WO2015/183969 and in particular Table 2 of WO2018/045308.

### Immunogenic Fragments

In one embodiment the isolated influenza HA stem polypeptide is a polypeptide comprising an immunogenic fragment of an influenza HA stem region. Suitably the isolated influenza HA stem polypeptide is a polypeptide consisting of an immunogenic fragment of an influenza HA stem region.

The immunogenic fragment of an influenza HA stem region of use in the present invention comprises, such as consists of, a fragment of a full length (such as native) influenza HA stem region which is capable of eliciting neutralising antibodies and/or a T cell response (such as a CD4 or CD8 T cell response) to influenza virus, preferably to influenza A virus, suitably a protective immune response (e.g. reducing partially or completely the severity of one or more symptoms and/or time over which one or more symptoms are experienced by a subject following infection, reducing the likelihood of developing an established infection after challenge and/or slowing progression of illness (e.g. extending survival)).

Suitably the immunogenic fragment of an influenza HA stem region comprises one or more epitopes from a full length influenza HA stem region, such as one, two or three or more epitopes.

The sequence of the immunogenic fragment of an influenza HA stem region may share 80% or greater, such as 90% or greater, such as 95% or greater, such as 98% or greater, such as 99% or greater, such as most suitably 100% identity with a corresponding sequence comprised within a full length influenza HA stem region, such as the sequences provided in SEQ ID NOs: 1-4, such as SEQ ID NO: 1, 2 or 4, more suitably SEQ ID NO: 2 or 4, most suitably SEQ ID NO: 2.

### Immunogenic Variants

In one embodiment the isolated influenza HA stem polypeptide is a polypeptide comprising an immunogenic variant of an influenza HA stem region. Suitably the isolated influenza HA stem polypeptide is a polypeptide consisting of an immunogenic variant of an influenza HA stem region.

The immunogenic variant of an influenza HA stem region of use in the present invention comprises, such as consists of, a variant of a full length (such as native) influenza HA stem region which is capable of eliciting neutralising antibodies and/or a T cell response (such as a CD4 or CD8 T cell response) to influenza virus, preferably to influenza A virus, suitably a protective immune response (e.g. reducing partially or completely the severity of one or more symptoms and/or time over which one or more symptoms are experienced by a subject following infection, reducing the likelihood of developing an established infection after challenge and/or slowing progression of illness (e.g. extending survival)).

The immunogenic variant of an influenza HA stem region may comprise, such as consist of, an amino acid sequence having at least 90%, such as at least 95%, such as at least 98%, such as at least 99%, such as 100% identity to the amino acid sequence set forth in SEQ ID NOs: 1-4, such as SEQ ID NO: 1, 2 or 4, more suitably SEQ ID NO: 2 or 4, most suitably SEQ ID NO: 2.

Suitably the immunogenic variant of an influenza HA stem region comprises one or more epitopes from a full length influenza HA stem region, such as one, two or three or more epitopes.

### Stability and Nanoparticles

For stable homotrimer assembly in its native environment, the influenza HA stem region requires fusion with the head region and the transmembrane domain. Arrangement in a homotrimer formation ensures antigenic conformational epitopes are presented. Accordingly, in one embodiment the isolated influenza HA stem polypeptide is a stable isolated influenza HA stem polypeptide, i.e. the polypeptide substantially retains its native conformation when administered to a subject.

The isolated influenza HA stem polypeptide may be synthetically stabilised (in the absence of head and transmembrane domains). Stabilisation may be achieved by helix stabilization, loop optimization, disulphide bond addition, and side-chain repacking (as disclosed in Corbett, 2019). Alternatively, or in addition, stabilisation may be achieved by providing the stem region in the form of a multimer, such as a homotrimer. Most suitably the isolated influenza HA stem polypeptide is provided in the form of a homotrimer, in particular a synthetically stabilised homotrimer.

The isolated influenza HA stem polypeptide may be provided ‘naked’, i.e. not bound to other materials. Alternatively, the isolated influenza HA stem polypeptide may be provided bound to one or more further agents. In a particular embodiment, the isolated influenza HA stem polypeptide is presented on the surface of nanoparticles, such as protein nanoparticles, such as those disclosed in Diaz et al 2018 including ferritin, lumazine and encapsulin. Protein nanoparticles present multiple faces on which antigenic proteins may be presented.

Ideally the isolated influenza HA stem polypeptide homotrimer is displayed on the surface of the protein nanoparticle, in particular on one or more of the individual faces of the protein nanoparticle, such as on all faces of the protein nanoparticle.

The protein nanoparticle may comprise one or more different isolated influenza HA stem polypeptides. In one embodiment the protein nanoparticle comprises one isolated influenza HA stem polypeptide (i.e. all isolated influenza HA stem polypeptides have an identical polypeptide sequence). In another embodiment, the protein nanoparticle comprises two different isolated influenza HA stem polypeptides. The two different isolated influenza HA stem polypeptides may each belong to e.g. group A1 and group A2 influenza, or to different subtypes within group A1 or A2, or to different strains within a subtype. A nanoparticle comprising only identical isolated influenza HA stem polypeptides may be referred to as a homodisplay particle. A nanoparticle comprising two or more different isolated influenza stem polypeptides from different influenza A strains or subtypes or groups, may be referred to as a heterodisplay particle. A nanoparticle comprising two or more different isolated influenza stem polypeptides belonging to different subtypes of influenza A, such as one from group A1 and one from group A2, for example H1 and H10, may be referred to as a heterotypic or more specifically a heterosubtypic particle. In one embodiment, nanoparticles are provided in a mixture of two or more populations of nanoparticles, each population displaying different influenza stem polypeptides from different strains and/or subtypes such as one from group A1 and one from group A2, for example H1 and H10.

In some embodiments, the protein nanoparticle displays at least one (for example two) influenza HA stem polypeptide(s) which comprises or consists of an amino acid sequence having at least 90%, 95%, 98% or 99% identity to the amino acid sequence set forth in any one of SEQ ID NO:1, SEQ ID NO: 2, SEQ ID NO: 3 or SEQ ID NO: 4.

Suitably the nanoparticle and the isolated influenza HA stem polypeptide are connected by a linker, such as a linker comprising the polypeptide sequence SGG, such as consisting of the polypeptide sequence SGG or GGSGG [SEQ ID NO: 12].

When provided in the form of a homotrimer, the isolated influenza HA stem polypeptide is most suitably displayed on self-assembling protein nanoparticles, such as most suitably ferritin nanoparticles, such as more suitably insect or bacterial ferritin nanoparticles, such as most suitably H. pylori ferritin nanoparticles or Trichoplusia ni nanoparticles (such as those disclosed in Corbett 2019, WO2013/044203, WO2015/183969, WO2018/045308, WO2018/005558 and Georgiev 2018).

Suitably, the ferritin nanoparticles comprise isolated influenza HA stem polypeptide connected to ferritin via a linker, such as a linker consisting of 1-10 amino acid residues, preferably 2-5 residues, such as a linker comprising the polypeptide sequence SGG, such as consisting of the polypeptide sequence SGG or GGSGG [SEQ ID NO: 12].

According to particular embodiments, the protein nanoparticles are bacterial ferritin nanoparticles, preferably H. pylori ferritin nanoparticles (such as those disclosed in Corbett, 2019, WO2013/044203, WO2015/183969 and WO2018/045308). Suitably the bacterial ferritin nanoparticles comprise or more suitably consist of a fusion protein of an isolated influenza HA stem polypeptide with bacterial ferritin, preferably H. pylori ferritin. In further embodiments, the bacterial ferritin nanoparticles comprise or more suitably consist of two or more fusion proteins of different isolated influenza HA stem polypeptides with bacterial ferritin, preferably H. pylori ferritin. In one embodiment, the two or more fusion proteins are provided within separate nanoparticles which are mixed together to form a mixed population of nanoparticles. In another embodiment, the two or more fusion proteins are co-expressed and co-assembled to display two or more different isolated influenza HA stem polypeptides on a single nanoparticle. For either mixed or co-assembled nanoparticles, the different influenza HA stem polypeptides are derived from different influenza A strains and/or subtypes, suitably from one or more influenza A Group 1 subtypes and/or one or more influenza A Group 2 subtypes, such as for example one from Group 1 and one from Group 2, such as one from H1 and one from H10. Suitably the HA stem and H. pylori ferritin are connected via a linker, suitably a linker comprising or consisting of the polypeptide sequence SGG.

According to alternative embodiments, the protein nanoparticles are insect ferritin nanoparticles, preferably Trichoplusia ni ferritin nanoparticles (such as those disclosed in WO2018/005558 and Georgiev 2018). Insect ferritin nanoparticles are formed from 12 heavy chain and 12 light chain proteins. Suitably the insect ferritin nanoparticles comprise or more suitably consist of a fusion protein of a first isolated influenza HA stem polypeptide with insect ferritin heavy chain, and a fusion protein of a second isolated influenza HA stem polypeptide with insect ferritin light chain, where the insect ferritin heavy and light chains are preferably from Trichoplusia ni. When co-expressed in a host cell, each fusion protein of influenza HA stem polypeptide with an insect ferritin heavy or light chain will self-assemble to form trimers, which in turn self-assemble to form nanoparticles displaying a plurality of influenza HA stem polypeptides. In one embodiment, the first and second isolated influenza HA stem polypeptides are the same, resulting in homodisplay insect ferritin nanoparticles. In other embodiments, the first and second influenza HA stem polypeptides are derived from one or more different strains or subtypes of influenza, resulting in heterodisplay insect ferritin nanoparticles. In a preferred embodiment, the first and second influenza HA stem polypeptides are derived from one or more influenza A Group 1 subtype and/or one or more influenza A Group 2 subtype, preferably one from Group 1 and one from Group 2, such as H1 and H10. Suitably the HA stem polypeptide and insect ferritin are connected via a linker, suitably a linker comprising or consisting of the polypeptide sequence GGSGG [SEQ ID NO: 12].

In some embodiments, the insect ferritin light and heavy chain are produced from a self-cleaving construct, such as a construct having at least 90%, such as at least 95%, such as at least 98%, such as at least 99%, such as 100% identity to the amino acid sequence set forth in SEQ ID NO: 13 (empty insect ferritin construct) further comprising two influenza HA stem polypeptides. In particular embodiments, the insect nanoparticles are produced from a self-cleaving construct such as a construct having at least 90%, such as at least 95%, such as at least 98%, such as at least 99%, such as 100% identity to the amino acid sequence set forth in SEQ ID NO: 14, 15 or 16, more particularly SEQ ID NO: 15. In certain embodiments the self-cleaving construct is cleaved at a furin cleavage site (F), such as an F2A cleavage site.

In some particular embodiments, the insect ferritin nanoparticles comprise a heavy chain comprising or consisting of a sequence having at least 90%, 95%, 98% or 99% or 100% identity to the amino acid sequence set forth SEQ ID NO:10, linked to an influenza HA stem polypeptide via a linker, and light chain comprising or consisting of a sequence having at least 90%, 95%, 98% or 99% or 100% identity to the amino acid sequence set forth SEQ ID NO:11, linked to an influenza HA stem polypeptide via a linker, wherein the HA stem polypeptides are different and at least one is selected from an amino acid sequence having at least 90%, 95%, 98% or 99% or 100% identity to the amino acid sequence set forth in any one of SEQ ID NO:1, SEQ ID NO: 2, SEQ ID NO: 3 or SEQ ID NO: 4.

Ferritin nanoparticles described herein can be produced in a suitable expression system, in particular a eukaryotic host cell, such as a mammalian cell e.g. a human cell such as HEK293T cell, non-human mammalian cell such as a CHO cell, or an insect cell. Ferritin nanoparticles displaying one or more influenza HA stem polypeptides are isolated/purified from the cells in which they are expressed.

### Sequence Alignments

Identity or homology with respect to a sequence is defined herein as the percentage of amino acid residues in the candidate sequence that are identical with the reference amino acid sequence after aligning the sequences and introducing gaps, if necessary, to achieve the maximum percent sequence identity, and not considering any conservative substitutions as part of the sequence identity.

Sequence identity can be determined by standard methods that are commonly used to compare the similarity in position of the amino acids of two polypeptides. Using a computer program such as BLAST or FASTA, two polypeptides are aligned for optimal matching of their respective amino acids (either along the full length of one or both sequences or along a pre-determined portion of one or both sequences). The programs provide a default opening penalty and a default gap penalty, and a scoring matrix such as PAM 250 (a standard scoring matrix; see Dayhoff, 1978) can be used in conjunction with the computer program. For example, the percent identity can then be calculated as: the total number of identical matches multiplied by 100 and then divided by the sum of the length of the longer sequence within the matched span and the number of gaps introduced into the shorter sequences in order to align the two sequences.

### Additional Antigens

The present invention may involve a plurality of antigenic components, for example with the objective to elicit a broad immune response to influenza virus. Consequently, more than one antigen may be present, more than one polynucleotide encoding an antigen may be present, one polynucleotide encoding more than one antigen may be present or a mixture of antigen(s) and polynucleotide(s) encoding antigen(s) may be present. Polysaccharides such as polysaccharide conjugates may also be present.

By the term antigen is meant a polypeptide which is capable of eliciting an immune response. Suitably the antigen comprises at least one B or T cell epitope. The elicited immune response may be an antigen specific B cell response, which produces neutralizing antibodies. The elicited immune response may be an antigen specific T cell response, which may be a systemic and/or a local response. The antigen specific T cell response may comprise a CD4+ T cell response, such as a response involving CD4+ T cells expressing a plurality of cytokines, e.g. IFNgamma, TNFalpha and/or IL2. Alternatively, or additionally, the antigen specific T cell response comprises a CD8+ T cell response, such as a response involving CD8+ T cells expressing a plurality of cytokines, e.g., IFNgamma, TNFalpha and/or IL2.

### Squalene Emulsion Adjuvant

The term ‘squalene emulsion adjuvant’ as used herein refers to a squalene containing oil-in-water emulsion adjuvant.

Squalene, is a branched, unsaturated terpenoid ([(CH₃)₂C[═CHCH₂CH₂C(CH₃)]₂═CHCH₂-]₂; C₃₀H₅₀; 2,6,10,15,19,23-hexamethyl-2,6,10,14,18,22-tetracosahexaene; CAS Registry Number 7683-64-9). Squalene is readily available from commercial sources or may be obtained by methods known in the art. Squalene shows good biocompatibility and is readily metabolised.

The squalene emulsion adjuvant may comprise one or more tocopherols, suitably wherein the weight ratio of squalene to tocopherol is 20 or less (i.e. 20 weight units of squalene or less per weight unit of tocopherol or, alternatively phrased, at least 1 weight unit of tocopherol per 20 weight units of squalene).

Any of the α, β, γ, δ, ε and/or ξ tocopherols can be used, but α-tocopherol (also referred to herein as alpha-tocopherol) is typically used. D-alpha-tocopherol and D/L-alpha-tocopherol can both be used. Tocopherols are readily available from commercial sources or may be obtained by methods known in the art. In some embodiments the squalene emulsion adjuvant contains alpha-tocopherol, especially D/L-alpha-tocopherol.

Squalene emulsion adjuvants will typically have a submicron droplet size. Droplet sizes below 200 nm are beneficial in that they can facilitate sterilisation by filtration. There is evidence that droplet sizes in the 80 to 200 nm range are of particular interest for potency, manufacturing consistency and stability reasons (Klucker, 2012; Shah, 2014; Shah, 2015; Shah, 2019). Suitably the squalene emulsion adjuvant has an average droplet size of less than 1 um, especially less than 500 nm and in particular less than 200 nm. Suitably the squalene emulsion adjuvant has an average droplet size of at least 50 nm, especially at least 80 nm, in particular at least 100 nm, such as at least 120 nm. The squalene emulsion adjuvant may have an average droplet size of 50 to 200 nm, such as 80 to 200 nm, especially 120 to 180 nm, in particular 140 to 180 nm, such as about 160 nm.

Uniformity of droplet sizes is desirable. A polydispersity index (Pdl) of greater than 0.7 indicates that the sample has a very broad size distribution and a reported value of 0 means that size variation is absent, although values smaller than 0.05 are rarely seen. Suitably the squalene emulsion adjuvant has a polydispersity of 0.5 or less, especially 0.3 or less, such as 0.2 or less.

The droplet size, as used herein, means the average diameter of oil droplets in an emulsion and can be determined in various ways e.g. using the techniques of dynamic light scattering and/or single-particle optical sensing, using an apparatus such as the Accusizer™ and Nicomp™ series of instruments available from Particle Sizing Systems (Santa Barbara, USA), the Zetasizer™ instruments from Malvern Instruments (UK), or the Particle Size Distribution Analyzer instruments from Horiba (Kyoto, Japan). See Light Scattering from Polymer Solutions and Nanoparticle Dispersions Schartl, 2007. Dynamic light scattering (DLS) is the preferred method by which droplet size is determined. The preferred method for defining the average droplet diameter is a Z-average i.e. the intensity-weighted mean hydrodynamic size of the ensemble collection of droplets measured by DLS. The Z-average is derived from cumulants analysis of the measured correlation curve, wherein a single particle size (droplet diameter) is assumed and a single exponential fit is applied to the autocorrelation function. Thus, references herein to average droplet size should be taken as an intensity-weighted average, and ideally the Z-average. Pdl values are easily provided by the same instrumentation which measures average diameter.

In order to maintain a stable submicron emulsion, one or more emulsifying agents (i.e. surfactants) are generally required. Surfactants can be classified by their ‘HLB’ (Griffin's hydrophile/lipophile balance), where a HLB in the range 1-10 generally means that the surfactant is more soluble in oil than in water, whereas a HLB in the range 10-20 means that the surfactant is more soluble in water than in oil. HLB values are readily available for many surfactants of interest or can be determined experimentally, e.g. polysorbate 80 has a HLB of 15.0 and TPGS has a HLB of 13 to 13.2. Sorbitan trioleate has a HLB of 1.8. When two or more surfactants are blended, the resulting HLB of the blend is typically calculated by the weighted average e.g. a 70/30 wt % mixture of polysorbate 80 and TPGS has a HLB of (15.0×0.70)+(13×0.30) i.e. 14.4. A 70/30 wt % mixture of polysorbate 80 and sorbitan trioleate has a HLB of (15.0×0.70)+(1.8×0.30) i.e. 11.04.

Surfactant(s) will typically be metabolisable (biodegradable) and biocompatible, being suitable for use as a pharmaceutical. The surfactant can include ionic (cationic, anionic or zwitterionic) and/or non-ionic surfactants. The use of only non-ionic surfactants is often desirable, for example due to their pH independence. The invention can thus use surfactants including, but not limited to:
the polyoxyethylene sorbitan ester surfactants (commonly referred to as the Tweens or polysorbates), such as polysorbate 20 and polysorbate 80, especially polysorbate 80;
copolymers of ethylene oxide (EO), propylene oxide (PO), and/or butylene oxide (BO), sold under the DOWFAX™, Pluronic™ (e.g. F68, F127 or L121 grades) or Synperonic™ tradenames, such as linear EO/PO block copolymers, for example poloxamer 407, poloxamer 401 and poloxamer 188;
octoxynols, which can vary in the number of repeating ethoxy (oxy-1,2-ethanediyl) groups, with octoxynol-9 (Triton X-100, or t-octylphenoxypolyethoxyethanol) being of particular interest;
(octylphenoxy)polyethoxyethanol (IGEPAL CA-630/NP-40);
phospholipids such as phosphatidylcholine (lecithin);
polyoxyethylene fatty ethers derived from lauryl, cetyl, stearyl and oleyl alcohols (known as Brij surfactants), such as polyoxyethylene 4 lauryl ether (Brij 30, Emulgen 104P), polyoxyethylene-9-lauryl ether and polyoxyethylene 12 cetyl/stearyl ether (Eumulgin™ B1, cetereth-12 or polyoxyethylene cetostearyl ether);
sorbitan esters (commonly known as the Spans), such as sorbitan trioleate (Span 85), sorbitan monooleate (Span 80) and sorbitan monolaurate (Span 20);
or tocopherol derivative surfactants, such as alpha-tocopherol-polyethylene glycol succinate (TPGS).

Many examples of pharmaceutically acceptable surfactants are known in the art e.g. see Handbook of Pharmaceutical Excipients 6th edition, 2009. Methods for selecting and optimising the choice of surfactant used in a squalene emulsion adjuvant are illustrated in Klucker, 2012. In general, the surfactant component has a HLB between 10 and 18, such as between 12 and 17, in particular 13 to 16. This can be typically achieved using a single surfactant or, in some embodiments, using a mixture of surfactants. Surfactants of particular interest include: poloxamer 401, poloxamer 188, polysorbate 80, sorbitan trioleate, sorbitan monooleate and polyoxyethylene 12 cetyl/stearyl ether either alone, in combination with each other or in combination with other surfactants. Especially of interest are polysorbate 80, sorbitan trioleate, sorbitan monooleate and polyoxyethylene 12 cetyl/stearyl ether either alone or in combination with each other. A particular surfactant of interest is polysorbate 80. A particular combination of surfactants of interest is polysorbate 80 and sorbitan trioleate. A further combination of surfactants of interest is sorbitan monooleate and polyoxyethylene cetostearyl ether.

In certain embodiments the squalene emulsion adjuvant comprises one surfactant, such as polysorbate 80. In some embodiments the squalene emulsion adjuvant comprises two surfactants, such as polysorbate 80 and sorbitan trioleate or sorbitan monooleate and polyoxyethylene cetostearyl ether. In other embodiments the squalene emulsion adjuvant comprises three or more surfactants, such as three surfactants.

If tocopherol is present, the weight ratio of squalene to tocopherol may be 20 or less, such as 10 or less. Suitably the weight ratio of squalene to tocopherol is 0.1 or more. Typically the weight ratio of squalene to tocopherol is 0.1 to 10, especially 0.2 to 5, in particular 0.3 to 3, such as 0.4 to 2. Suitably, the weight ratio of squalene to tocopherol is 0.72 to 1.136, especially 0.8 to 1, in particular 0.85 to 0.95, such as 0.9.

If surfactant is present, typically the weight ratio of squalene to surfactant is 0.73 to 6.6, especially 1 to 5, in particular 1.2 to 4. Suitably, the weight ratio of squalene to surfactant is 1.71 to 2.8, especially 2 to 2.4, in particular 2.1 to 2.3, such as 2.2.

The amount of squalene in a single dose, such as a human dose, of squalene emulsion adjuvant is typically at least 1.2 mg. Generally, the amount of squalene in a single dose, such as a human dose, of squalene emulsion adjuvant is 50 mg or less. The amount of squalene in a single dose, such as a human dose, of squalene emulsion adjuvant may be 1.2 to 20 mg, in particular 1.2 to 15 mg. The amount of squalene in a single dose, such as a human dose, of squalene emulsion adjuvant may be 1.2 to 2 mg, 2 to 4 mg, 4 to 8 mg or 8 to 12.1 mg. For example, the amount of squalene in a single dose, such as a human dose, of squalene emulsion adjuvant may be 1.21 to 1.52 mg, 2.43 to 3.03 mg, 4.87 to 6.05 mg or 9.75 to 12.1 mg.

If tocopherol is present, the amount of tocopherol in a single dose, such as a human dose, of squalene emulsion adjuvant is typically at least 1.3 mg. Generally, the amount of tocopherol in a single dose, such as a human dose, of squalene emulsion adjuvant is 55 mg or less. The amount of tocopherol in a single dose, such as a human dose, of squalene emulsion adjuvant may be 1.3 to 22 mg, in particular 1.3 to 16.6 mg. The amount of tocopherol in a single dose, such as a human dose, of squalene emulsion adjuvant may be 1.3 to 2 mg, 2 to 4 mg, 4 to 8 mg or 8 to 13.6 mg. For example, the amount of tocopherol in a single dose, such as a human dose, of squalene emulsion adjuvant may be 1.33 to 1.69 mg, 2.66 to 3.39 mg, 5.32 to 6.77 mg or 10.65 to 13.53 mg.

If surfactant is present, the amount of surfactant in a single dose, such as a human dose, of squalene emulsion adjuvant is typically at least 0.4 mg. Generally, the amount of surfactant in a single dose, such as a human dose, of squalene emulsion adjuvant is 18 mg or less. The amount of surfactant in a single dose, such as a human dose, of squalene emulsion adjuvant may be 0.4 to 9.5 mg, in particular 0.4 to 7 mg. The amount of surfactant in a single dose, such as a human dose, of squalene emulsion adjuvant may be 0.4 to 1 mg, 1 to 2 mg, 2 to 4 mg or 4 to 7 mg. For example, the amount of surfactant in a single dose, such as a human dose, of squalene emulsion adjuvant may be 0.54 to 0.71 mg, 1.08 to 1.42 mg, 2.16 to 2.84 mg or 4.32 to 5.68 mg.

In particular embodiments, the squalene emulsion adjuvant comprises or consists of:
squalene (10.69 mg)+polysorbate 80 (4.86 mg)+alpha-tocopherol (11.86 mg) per human dose (irrespective of the volume); or
squalene (5.34 mg)+polysorbate 80 (2.43 mg)+alpha-tocopherol (5.93 mg) per human dose (irrespective of the volume).

In certain embodiments the squalene emulsion adjuvant may consist essentially of squalene, surfactant and water. In certain other embodiments the squalene emulsion adjuvant may consist essentially of squalene, tocopherol, surfactant and water. Squalene emulsion adjuvants may contain additional components as desired or required depending upon the intended final presentation and vaccination strategy, such as buffers and/or tonicity modifying agents, for example modified phosphate buffered saline (disodium phosphate, potassium biphosphate, sodium chloride and potassium chloride).

High pressure homogenization (HPH or microfluidisation) may be applied to yield squalene emulsion adjuvants comprising tocopherol which demonstrate uniformly small droplet sizes and long-term stability (see EP0868918 and WO2006/100109). Briefly, oil phase composed of squalene and tocopherol may be formulated under a nitrogen atmosphere. Aqueous phase is prepared separately, typically composed of water for injection or phosphate buffered saline, and polysorbate 80. Oil and aqueous phases are combined, such as at a ratio of 1:9 (volume of oil phase to volume of aqueous phase) before homogenisation and microfluidisation, such as by a single pass through an in-line homogeniser and three passes through a microfluidiser (at around 15000 psi). The resulting emulsion may then be sterile filtered, for example through two trains of two 0.5/0.2 um filters in series (i.e. 0.5/0.2/0.5/0.2), see WO2011/154444. Operation is desirably undertaken under an inert atmosphere, e.g. nitrogen. Positive pressure may be applied, see WO2011/154443.

International patent application WO2020160080 and Lodaya, 2019 describe squalene emulsion adjuvants comprising tocopherol which are self-emulsifying adjuvant systems (SEAS) and their manufacture.

In one embodiment, the squalene emulsion adjuvant is not AF03 (as disclosed in Klucker et al. 2021). More suitably, the squalene emulsion adjuvant does not comprise polyoxyethylene cetostearyl ether and/or mannitol and/or sorbitan oleate.

### Assays

The in vitro efficacy of vaccines which target the head region may be established by assays which investigate whether or not the vaccine prevents influenza virus from binding to target cells. An example of such an assay is the hemagglutination inhibition (HAI) assay, which is considered to be the gold standard in the field and which provides a correlate of protection in vivo. However, vaccines which target the stem region, while being potentially protective, may not prevent influenza virus from binding to target cells. The above assays are therefore inappropriate for investigating the efficacy of a vaccine targeting the stem region.

Suitable assays for investigating the efficacy of a vaccine targeting the stem region which has been administered to mice are as follows. Implementations of these assays are used in the examples provided herein.

### Anti-HA IgG Antibodies by ELISA

Quantification of mouse anti-HA IgG antibodies are performed by ELISA using HA antigen (full length or stem only) as coating. The plates are then incubated. Diluted sera are added to the coated plates and incubated. The plates are washed prior to the adding of diluted peroxidase conjugated goat anti-mouse IgG. The reaction is stopped with H₂SO₄ and optical densities are read. The titers are expressed as ELISA Units Titers.

### Stem Specific T Cell Frequencies

Spleens are collected and cell suspensions are prepared. The splenic cell suspensions are filtered, harvested and centrifuged. Fresh splenocytes are then plated in the presence of an overlapping peptide pool covering the sequence of stem protein. Following stimulation, cells are washed and stained with anti-CD16/32, anti-CD4-V450 and anti-CD8-PerCp-Cy5.5 antibodies. Living/dead cell stain is added. Cells are permeabilized and stained with anti-IL2-FITC, anti-IFNγ-APC and anti-TNFα-PE antibodies. Stained cells are analyzed by flow cytometry.

### Neutralization Antibody Titers

Mouse sera are diluted and incubated in the presence of reporter influenza virus. After incubation, the serum-virus mix is added to cell culture. Influenza-positive cells are analysed and quantified by flow cytometry. Titers are expressed as 50% neutralization titers (IC50), corresponding to reduction titers calculated by regression analysis of the inverse dilution of serum that provides 50% cell infected reduction compared to control wells (virus only, no serum).

More specific implementations of the above assays are detailed in the examples. These more specific assays may also be used for investigating the efficacy of a vaccine targeting the stem region.

### Subjects

The present invention is generally intended for mammalian subjects, in particular human subjects. The subject may be a wild or domesticated animal. Mammalian subjects include for example cats, dogs, pigs, sheep, horses or cattle. In one embodiment the invention, the subject is human.

The subject to be treated using the method of the invention may be of any age.

In one embodiment the subject is a human infant (up to 12 months of age). In one embodiment the subject is a human child (less than 18 years of age). In one embodiment the subject is an adult human (aged 18-59). In one embodiment the subject is an older human (aged 60 or greater).

Doses administered to younger children, such as less than 12 years of age, may be reduced relative to an equivalent adult dose, such as by 50%.

The methods of the invention are suitably intended for prophylaxis, i.e. for administration to a subject which is not infected with influenza virus.

### Formulation and Administration

The isolated influenza HA stem polypeptide and squalene emulsion adjuvant may be administered as a formulation containing the isolated influenza HA stem polypeptide and squalene emulsion adjuvant (‘co-formulation’ or ‘co-formulated’). Alternatively the isolated influenza HA stem polypeptide and squalene emulsion adjuvant may be administered as a first formulation containing the isolated influenza HA stem polypeptide and a second formulation containing the squalene emulsion adjuvant (‘separate formulation’ or ‘separately formulated’). When separately formulated, the isolated influenza HA stem polypeptide and squalene emulsion adjuvant may be administered through the same or different routes, to the same or different locations, and at the same or different times.

The isolated influenza HA stem polypeptide and squalene emulsion adjuvant may be administered via various suitable routes, including parenteral, such as intramuscular or subcutaneous administration. The isolated influenza HA stem polypeptide and squalene emulsion adjuvant may be administered via different routes. Suitably the isolated influenza HA stem polypeptide and squalene emulsion adjuvant are administered via the same route, in particular intramuscularly.

When administered as separate formulations, the isolated influenza HA stem polypeptide and squalene emulsion adjuvant are desirably administered to locations with sufficient spatial proximity such that the adjuvant effect is adequately maintained. For example, spatial proximity is sufficient to maintain at least 50%, especially at least 75% and in particular at least 90% of the adjuvant effect seen with administration at to the same location. The adjuvant effect seen with administration to the same location is defined as the level of increase observed as a result of administration of the isolated influenza HA stem polypeptide and squalene emulsion adjuvant to the same location compared with administration of the isolated influenza HA stem polypeptide alone. The isolated influenza HA stem polypeptide and squalene emulsion adjuvant are desirably administered to a location draining to the same lymph node, such as to the same limb, in particular to the same muscle.

Suitably the isolated influenza HA stem polypeptide and squalene emulsion adjuvant are administered intramuscularly to the same muscle. In certain embodiments, the isolated influenza HA stem polypeptide and squalene emulsion adjuvant are administered to the same location.

The spatial separation of administration locations may be at least 5 mm, such as at least 1 cm. The spatial separation of administration locations may be less than 10 cm, such as less than 5 cm apart.

When administered as separate formulations, the isolated influenza HA stem polypeptide and squalene emulsion adjuvant are desirably administered with sufficient temporal proximity such that the adjuvant effect is adequately maintained. For example, temporal proximity is sufficient to maintain at least 50%, especially at least 75% and in particular at least 90% of the adjuvant effect seen with administration at the same time. The adjuvant effect seen with administration at the same time is defined as the level of increase observed as a result of administration at (essentially) the same time compared with administration of the isolated influenza HA stem polypeptide without squalene emulsion adjuvant.

When administered as separate formulations, isolated influenza HA stem polypeptide and squalene emulsion adjuvant may be administered within 12 hours. Suitably the isolated influenza HA stem polypeptide and squalene emulsion adjuvant are administered within 6 hours, especially within 2 hours, in particular within 1 hour, such as within 30 minutes and especially within 15 minutes (e.g. within 5 minutes).

The delay between administration of the isolated influenza HA stem polypeptide and squalene emulsion adjuvant may be at least 5 seconds, such as 10 seconds, and in particular at least 30 seconds.

When administered as separate formulations, if the isolated influenza HA stem polypeptide and squalene emulsion adjuvant are administered with a delay, the isolated influenza HA stem polypeptide may be administered first and the squalene emulsion adjuvant administered second. Alternatively, the squalene emulsion adjuvant is administered first and the isolated influenza HA stem polypeptide administered second. Appropriate temporal proximity may depend on the order or administration.

Desirably, the isolated influenza HA stem polypeptide and squalene emulsion adjuvant are administered without intentional delay (accounting for the practicalities of multiple administrations).

In addition to co-formulated or separately formulated presentations of the isolated influenza HA stem polypeptide and squalene emulsion adjuvant for direct administration, the isolated influenza HA stem polypeptide and squalene emulsion adjuvant may initially be provided in various forms which facilitate manufacture, storage and distribution. For example, certain components may have limited stability in liquid form, certain components may not be amendable to drying, certain components may be incompatible when mixed (either on a short- or long-term basis). Independent of whether the isolated influenza HA stem polypeptide and squalene emulsion are co-formulated at administration, they may be provided in separate containers the contents of which are subsequently combined. The skilled person will appreciate that many possibilities exist, although it is generally desirable to have a limited number of containers and limited number of required steps to prepare the final co-formulation or separate formulations for administration.

The isolated influenza HA stem polypeptide may be provided in liquid or dry (e.g. lyophilised) form. The preferred form will depend on factors such as the precise nature of the isolated influenza HA stem polypeptide, e.g. if the isolated influenza HA stem polypeptide is amenable to drying, or other components which may be present.

The squalene emulsion adjuvant is provided in liquid form.

The invention provides a composition comprising an isolated influenza HA stem polypeptide and a squalene emulsion adjuvant. Typically the isolated influenza HA stem polypeptide and squalene emulsion adjuvant are provided as a liquid co-formulation. A liquid co-formulation enables convenient administration at the point of use.

The isolated influenza HA stem polypeptide and squalene emulsion adjuvant may be provided in separate containers. The invention therefore provides an isolated influenza HA stem polypeptide for use with a squalene emulsion adjuvant. Also provided is a squalene emulsion adjuvant for use with an isolated influenza HA stem polypeptide. Further provided is a kit comprising:
(i) a first container comprising an isolated influenza HA stem polypeptide; and
(ii) a second container comprising a squalene emulsion adjuvant.

The isolated influenza HA stem polypeptide may be in liquid form and the squalene emulsion adjuvant may be in liquid form. In such cases the contents of the first and second containers may be intended for combination to provide a co-formulation for administration. Alternatively, the contents of each container may be intended for separate administration as the first and second formulations.

The isolated influenza HA stem polypeptide may be in dry form and the squalene emulsion adjuvant may be in liquid form. In such cases the contents of the first and second containers may be intended for combination to provide a co-formulation for administration. Alternatively, the isolated influenza HA stem polypeptide may be intended to be reconstituted prior to the contents of each container being used for separate administration as the first and second formulations.

The precise composition of liquid used for reconstitution will depend on both the contents of a container being reconstituted and the subsequent use of the reconstituted contents e.g. if they are intended for administration directly or may be combined with other components prior to administration. A composition (such as those containing isolated influenza HA stem polypeptide or squalene emulsion adjuvant) intended for combination with other compositions prior to administration need not itself have a physiologically acceptable pH or a physiologically acceptable tonicity; a formulation intended for administration should have a physiologically acceptable pH and should have a physiologically acceptable osmolality.

The pH of a liquid preparation is adjusted in view of the components of the composition and necessary suitability for administration to the human subject. The pH of a formulation is generally at least 4, especially at least 5, in particular at least 5.5 such as at least 6. The pH of a formulation is generally 9 or less, especially 8.5 or less, in particular 8 or less, such as 7.5 or less. The pH of a formulation may be 4 to 9, especially 5 to 8.5, in particular 5.5 to 8, such as 6.5 to 7.4 (e.g. 6.5 to 7.1).

For parenteral administration, solutions should have a physiologically acceptable osmolality to avoid excessive cell distortion or lysis. A physiologically acceptable osmolality will generally mean that solutions will have an osmolality which is approximately isotonic or mildly hypertonic. Suitably the formulations for administration will have an osmolality of 250 to 750 mOsm/kg, especially 250 to 550 mOsm/kg, in particular 270 to 500 mOsm/kg, such as 270 to 400 mOsm/kg. Osmolality may be measured according to techniques known in the art, such as by the use of a commercially available osmometer, for example the Advanced® Model 2020 available from Advanced Instruments Inc. (USA).

Liquids used for reconstitution will be substantially aqueous, such as water for injection, phosphate buffered saline and the like. As mentioned above, the requirement for buffer and/or tonicity modifying agents will depend on the on both the contents of the container being reconstituted and the subsequent use of the reconstituted contents. Buffers may be selected from acetate, citrate, histidine, maleate, phosphate, succinate, tartrate and TRIS. The buffer may be a phosphate buffer such as Na/Na₂PO₄, Na/K₂PO₄ or K/K₂PO₄.

Suitably, the formulations used in the present invention have a dose volume of between 0.05 ml and 1 ml, such as between 0.1 and 0.6 ml, in particular a dose volume of 0.45 to 0.55 ml, such as 0.5 ml. The volumes of the compositions used may depend on the subject, delivery route and location, with smaller doses being given by the intradermal route or if both the isolated influenza HA stem polypeptide and squalene emulsion adjuvant are delivered to the same location. A typical human dose for administration through routes such as intramuscular, is in the region of 200 ul to 750 ml, such as 400 to 600 ul, in particular about 500 ul, such as 500 ul.

If two liquids are intended to be combined, for example for co-formulation if the isolated influenza HA stem polypeptide is in liquid form and the squalene emulsion adjuvant is in liquid form, the volume of each liquid may be the same or different. Volumes for combination will typically be in the range of 10:1 to 1:10, such as 2:1 to 1:2. Suitably the volume of each liquid will be substantially the same, such as the same. For example a 250 ul volume of the isolated influenza HA stem polypeptide in liquid form may be combined with a 250 ul volume squalene emulsion adjuvant in liquid form to provide a co-formulation dose with a 500 ul volume, each of the isolated influenza HA stem polypeptide and squalene emulsion adjuvant being diluted 2-fold during the combination.

Squalene emulsion adjuvants may therefore be prepared as a concentrate with the expectation of dilution by a liquid isolated influenza HA stem polypeptide containing composition prior to administration. For example, squalene emulsion adjuvant may be prepared at double-strength with the expectation of dilution by an equal volume of isolated influenza HA stem polypeptide containing composition prior to administration.

The concentration of squalene at administration may be in the range 0.8 to 100 mg per ml, especially 1.2 to 48.4 mg per ml.

The isolated influenza HA stem polypeptide and squalene emulsion adjuvant, whether intended for co-formulation or separate formulation, may be provided in the form of various physical containers such as vials or pre-filled syringes.

In some embodiments the isolated influenza HA stem polypeptide, squalene emulsion adjuvant or kit comprising the isolated influenza HA stem polypeptide and squalene emulsion adjuvant is provided in the form of a single dose. In other embodiments the isolated influenza HA stem polypeptide, squalene emulsion adjuvant or kit comprising isolated influenza HA stem polypeptide and squalene emulsion adjuvant is provided in multidose form such containing 2, 5 or 10 doses. Multidose forms, such as those comprising 10 doses, may be provided in the form of a plurality of containers with single doses of one part (e.g. the isolated influenza HA stem polypeptide) and a single container with multiple doses of the second part (e.g. squalene emulsion adjuvant) or may be provided in the form of a single container with multiple doses of one part (e.g. the isolated influenza HA stem polypeptide) and a single container with multiple doses of the second part (e.g. the squalene emulsion adjuvant).

It is common where liquids are to be transferred between containers, such as from a vial to a syringe, to provide ‘an overage’ which ensures that the full volume required can be conveniently transferred. The level of overage required will depend on the circumstances but excessive overage should be avoided to reduce wastage and insufficient overage may cause practical difficulties. Overages may be of the order of 20 to 100 ul per dose, such as 30 ul or 50 ul. For example, a typical 10 dose container of doubly concentrated squalene emulsion adjuvant (250 ul per dose) may contain around 2.85 to 3.25 ml of squalene emulsion adjuvant.

Stabilisers may be present. Stabilisers may be of particular relevance where multidose containers are provided as doses of the final formulation(s) may be administered to subjects over a period of time.

The isolated influenza HA stem polypeptide and squalene emulsion adjuvant in liquid form may be provided in the form of a multichamber syringe. The use of multi-chamber syringes provides a convenient method for the separate sequential administration of the isolated influenza HA stem polypeptide and squalene emulsion adjuvant. Multi-chamber syringes may be configured to provide concurrent but separate delivery of the isolated influenza HA stem polypeptide and squalene emulsion adjuvant, or they may be configured to provide sequential delivery (in either order).

In other configurations of multichambered syringes, the isolated influenza HA stem polypeptide may be provided in dry form (e.g. freeze-dried) in one chamber and reconstituted by the squalene emulsion adjuvant contained in the other chamber before administration.

Examples of multi-chamber syringes may be found in disclosures such as WO2016/172396, although a range of other configurations are possible.

Formulations are sterile.

Approaches for establishing strong and lasting immunity often include repeated immunisation, i.e. boosting an immune response by administration of one or more further doses. Such further administrations may be performed with the same immunogenic compositions (homologous boosting) or with different immunogenic compositions (heterologous boosting). The present invention may be applied as part of a homologous or heterologous prime/boost regimen, as either the priming or a/the boosting immunisation.

Administration of the isolated influenza HA stem polypeptide and squalene emulsion adjuvant may therefore be part of a multi-dose administration regime. For example, the isolated influenza HA stem polypeptide and squalene emulsion adjuvant may be provided as a priming dose in a multidose regime, especially a two- or three-dose regime, in particular a two-dose regime. The isolated influenza HA stem polypeptide and squalene emulsion adjuvant may be provided as a boosting dose in a multidose regime, especially a two- or three-dose regime, such as a two-dose regime.

Priming and boosting doses may be homologous or heterologous. Consequently, the isolated influenza HA stem polypeptide and squalene emulsion adjuvant may be provided as a priming dose and boosting dose(s) in a homologous multidose regime, especially a two- or three-dose regime, in particular a two-dose regime. Alternatively, the isolated influenza HA stem polypeptide and squalene emulsion adjuvant may be provided as a priming dose or boosting dose in a heterologous multidose regime, especially a two- or three-dose regime, in particular a two-dose regime, and the boosting dose(s) may be different (e.g. isolated influenza HA stem polypeptide; or an alternative antigen presentation such as protein or virally vectored antigen—with or without adjuvant, such as squalene emulsion adjuvant).

The time between doses may be two weeks to six months, such as three weeks to three months. Periodic longer-term booster doses may be also be provided, such as every 2 to 10 years.

The squalene emulsion adjuvant may be administered to a subject separately from the isolated influenza HA stem polypeptide, or the adjuvant may be combined, either during manufacturing or extemporaneously, with the isolated influenza HA stem polypeptide to provide an immunogenic composition for combined administration.

Consequently, there is provided a method for the preparation of an immunogenic composition comprising a squalene emulsion adjuvant and isolated influenza HA stem polypeptide, said method comprising the steps of:
(i) preparing a squalene emulsion adjuvant;
(ii) mixing the squalene emulsion adjuvant with the isolated influenza HA stem polypeptide.

Also provided a method for the preparation of an immunogenic composition comprising a squalene emulsion adjuvant and isolated influenza HA stem polypeptide, said method comprising the steps of:
(i) preparing an isolated influenza HA stem polypeptide;
(ii) mixing the isolated influenza HA stem polypeptide with a squalene emulsion adjuvant.

To limit undesired degradation, squalene emulsions should generally be stored with limited exposure to oxygen e.g. in containers with limited headspace and/or by storage under nitrogen.

Throughout the specification, including the claims, where the context permits, the term “comprising” and variants thereof such as “comprises” are to be interpreted as including the stated element (e.g., integer) or elements (e.g., integers) without necessarily excluding any other elements (e.g., integers). Thus a composition “comprising” X may consist exclusively of X or may include something additional e.g. X+Y.

The word “substantially” does not exclude “completely” e.g. a composition which is “substantially free” from Y may be completely free from Y. Where necessary, the word “substantially” may be omitted from the definition of the invention.

The term “about” in or “approximately” in relation to a numerical value x is optional and means, for example, x±10% of the given figure, such as x±5% of the given figure.

As used herein, the singular forms “a,” “an” and “the” include plural references unless the content clearly dictates otherwise.

Unless specifically stated, a process comprising a step of mixing two or more components does not require any specific order of mixing. Thus components can be mixed in any order.

Where there are three components then two components can be combined with each other, and then the combination may be combined with the third component, etc.

The present invention is also characterised by the following embodiments as set out in numbered paragraphs 1-76 below:

1. A method of eliciting an immune response in a subject, the method comprising administering to the subject (i) at least one isolated influenza HA stem polypeptide and (ii) a squalene emulsion adjuvant.

2. A method of adjuvanting the immune response of a subject to an isolated influenza HA stem polypeptide, the method comprising administering to the subject a squalene emulsion adjuvant.

3. A squalene emulsion adjuvant for use in eliciting an immune response in a subject by administration with an isolated influenza HA stem polypeptide.

4. An isolated influenza HA stem polypeptide, for use in eliciting an immune response in a subject by administration with a squalene emulsion adjuvant.

5. Use of a squalene emulsion adjuvant in the manufacture of a medicament for use in eliciting an immune response in a subject by administration with an isolated influenza HA stem polypeptide.

6. Use of an isolated influenza HA stem polypeptide in the manufacture of a medicament for use in eliciting an immune response in a subject by administration with a squalene emulsion adjuvant.

7. A kit comprising:
(i) a first container comprising an isolated influenza HA stem polypeptide; and
(ii) a second container comprising a squalene emulsion adjuvant.

8. The kit according to paragraph 7, further comprising instructions for combining a single dose of the isolated influenza HA stem polypeptide with a single dose of the squalene emulsion adjuvant to produce an immunogenic composition prior to administration of the immunogenic composition to a subject.

9. An immunogenic composition comprising: (i) an isolated influenza HA stem polypeptide, and (ii) a squalene emulsion adjuvant.

10. Use of (i) an isolated influenza HA stem polypeptide, and (ii) a squalene emulsion adjuvant, in the manufacture of a medicament.

11. The method, emulsion, isolated influenza HA stem polypeptide, use, kit or composition according to any one of paragraphs 1 to 10, wherein the isolated influenza HA stem polypeptide has been synthetically stabilised.

12. The method, emulsion, isolated influenza HA stem polypeptide, use, kit or composition according to any one of paragraphs 1 to 11, wherein the isolated influenza HA stem polypeptide is presented in the form of a homotrimer.

13. The method, emulsion, isolated influenza HA stem polypeptide, use, kit or composition according to any one of paragraphs 1 to 12, wherein the isolated influenza HA stem polypeptide is presented on a protein nanoparticle.

14. The method, emulsion, isolated influenza HA stem polypeptide, use, kit or composition according to paragraph 13, wherein the protein nanoparticle is ferritin.

15. The method, emulsion, isolated influenza HA stem polypeptide, use, kit or composition according to paragraph 14, wherein the ferritin is selected from bacterial or insect ferritin.

16. The method, emulsion, isolated influenza HA stem polypeptide, use, kit or composition according to paragraph 15, wherein the ferritin is bacterial ferritin such as H. pylori ferritin.

17. The method, emulsion, isolated influenza HA stem polypeptide, use, kit or composition according to paragraph 16, wherein the ferritin is insect ferritin such as Trichoplusia ni ferritin.

18. The method, emulsion, isolated influenza HA stem polypeptide, use, kit or composition according to any one of paragraphs 13 to 17, wherein the protein nanoparticle and the isolated influenza HA stem polypeptide are connected by a linker.

19. The method, emulsion, isolated influenza HA stem polypeptide, use, kit or composition according to paragraph 18, wherein the linker consists of 1 to 10 residues.

20. The method, emulsion, isolated influenza HA stem polypeptide, use, kit or composition according to paragraph 19, wherein the linker consists of 2 to 5 residues.

21. The method, emulsion, isolated influenza HA stem polypeptide, use, kit or composition according to any one of paragraphs 18 to 20, wherein the linker comprises the polypeptide sequence SGG.

22. The method, emulsion, isolated influenza HA stem polypeptide, use, kit or composition according to paragraph 21, wherein the linker consists of the polypeptide sequence SGG or the polypeptide sequence GGSGG [SEQ ID NO: 12].

23. The method, emulsion, isolated influenza HA stem polypeptide, use, kit or composition according to any one of paragraphs 1 to 22, wherein the isolated influenza HA stem polypeptide is a polypeptide comprising a full length influenza HA stem region.

24. The method, emulsion, isolated influenza HA stem polypeptide, use, kit or composition according to paragraph 23, wherein the isolated influenza HA stem polypeptide is a polypeptide consisting of a full length influenza HA stem region.

25. The method, emulsion, isolated influenza HA stem polypeptide, use, kit or composition according to any one of paragraphs 1 to 22, wherein the isolated influenza HA stem polypeptide is a polypeptide comprising an immunogenic fragment of an influenza HA stem region.

26. The method, emulsion, isolated influenza HA stem polypeptide, use, kit or composition according to paragraph 25, wherein the isolated influenza HA stem polypeptide is a polypeptide consisting of an immunogenic fragment of an influenza HA stem region.

27. The method, emulsion, isolated influenza HA stem polypeptide, use, kit or composition according to any one of paragraphs 1 to 22, wherein the isolated influenza HA stem polypeptide is a polypeptide comprising an immunogenic variant of an influenza HA stem region.

28. The method, emulsion, isolated influenza HA stem polypeptide, use, kit or composition according to paragraph 27 wherein the isolated influenza HA stem polypeptide is a polypeptide consisting of an immunogenic variant of an influenza HA stem region.

29. The method, emulsion, isolated influenza HA stem polypeptide, use, kit or composition according to any one of paragraphs 1 to 22, wherein the isolated influenza HA stem polypeptide comprises an amino acid sequence having at least 90% identity to the amino acid sequence set forth in any one of SEQ ID NO:1, SEQ ID NO: 2, SEQ ID NO: 3 or SEQ ID NO: 4.

30. The method, emulsion, isolated influenza HA stem polypeptide, use, kit or composition according to paragraph 29, wherein the isolated influenza HA stem polypeptide consists of an amino acid sequence having at least 90% identity to the amino acid sequence set forth in any one of SEQ ID NO:1, SEQ ID NO: 2, SEQ ID NO: 3 or SEQ ID NO: 4.

31. The method, emulsion, isolated influenza HA stem polypeptide, use, kit or composition according to paragraph 29, wherein the isolated influenza HA stem polypeptide comprises an amino acid sequence having at least 95% identity to the amino acid sequence set forth in any one of SEQ ID NO:1, SEQ ID NO: 2, SEQ ID NO: 3 or SEQ ID NO: 4.

32. The method, emulsion, isolated influenza HA stem polypeptide, use, kit or composition according to paragraph 30, wherein the isolated influenza HA stem polypeptide consists of an amino acid sequence having at least 95% identity to the amino acid sequence set forth in any one of SEQ ID NO:1, SEQ ID NO: 2, SEQ ID NO: 3 or SEQ ID NO: 4.

33. The method, emulsion, isolated influenza HA stem polypeptide, use, kit or composition according to paragraph 31, wherein the isolated influenza HA stem polypeptide comprises an amino acid sequence having at least 98% identity to the amino acid sequence set forth in any one of SEQ ID NO:1, SEQ ID NO: 2, SEQ ID NO: 3 or SEQ ID NO: 4.

34. The method, emulsion, isolated influenza HA stem polypeptide, use, kit or composition according to paragraph 32, wherein the isolated influenza HA stem polypeptide consists of an amino acid sequence having at least 98% identity to the amino acid sequence set forth in any one of SEQ ID NO:1, SEQ ID NO: 2, SEQ ID NO: 3 or SEQ ID NO: 4.

35. The method, emulsion, isolated influenza HA stem polypeptide, use, kit or composition according to paragraph 33, wherein the isolated influenza HA stem polypeptide comprises an amino acid sequence having at least 99% identity to the amino acid sequence set forth in any one of SEQ ID NO:1, SEQ ID NO: 2, SEQ ID NO: 3 or SEQ ID NO: 4.

36. The method, emulsion, isolated influenza HA stem polypeptide, use, kit or composition according to paragraph 34, wherein the isolated influenza HA stem polypeptide consists of an amino acid sequence having at least 99% identity to the amino acid sequence set forth in any one of SEQ ID NO:1, SEQ ID NO: 2, SEQ ID NO: 3 or SEQ ID NO: 4.

37. The method, emulsion, isolated influenza HA stem polypeptide, use, kit or composition according to paragraph 35, wherein the isolated influenza HA stem polypeptide comprises the amino acid sequence set forth in any one of SEQ ID NO:1, SEQ ID NO: 2, SEQ ID NO: 3 or SEQ ID NO: 4.

38. The method, emulsion, isolated influenza HA stem polypeptide, use, kit or composition according to paragraph 36, wherein the isolated influenza HA stem polypeptide consists of the amino acid sequence set forth in any one of SEQ ID NO:1, SEQ ID NO: 2, SEQ ID NO: 3 or SEQ ID NO: 4.

39. The method, emulsion, isolated influenza HA stem polypeptide, use, kit or composition according to any one of paragraphs 29 to 38, wherein the sequence is that set forth in SEQ ID NO: 2.

40. The method, emulsion, isolated influenza HA stem polypeptide, use, kit or composition according to paragraph 39, wherein the isolated influenza HA stem polypeptide comprises the amino acid sequence set forth in SEQ ID NO:2.

41. The method, emulsion, isolated influenza HA stem polypeptide, use, kit or composition according to paragraph 40, wherein the isolated influenza HA stem polypeptide consists of the amino acid sequence set forth in SEQ ID NO:2.

42. The method, emulsion, isolated influenza HA stem polypeptide, use, kit or composition according any one of paragraphs 1 to 41, wherein the isolated influenza HA stem polypeptide is 400 residues or fewer in length, especially 300 residues or fewer, in particular 250 residues or fewer, such as 220 residues or fewer.

43. The method, emulsion, isolated influenza HA stem polypeptide, use, kit or composition according any one of paragraphs 1 to 42, wherein the isolated influenza HA stem polypeptide is 130 residues or more in length, especially 160 residues or more, in particular 180 residues or more, such as 190 residues or more.

44. The method, emulsion, isolated influenza HA stem polypeptide, use, kit or composition according any one of paragraphs 1 to 43, wherein the isolated influenza HA stem polypeptide is 130 to 400 residues in length, especially 160 to 300, in particular 180 to 250, such as 190 to 220.

45. The method, emulsion, isolated influenza HA stem polypeptide, use, kit or composition according to any one of paragraphs 1 to 44, wherein a single dose of isolated influenza HA stem polypeptide is 0.001 to 1000 ug, especially 0.01 to 100 ug, in particular 0.1 to 50 ug.

46. The method, emulsion, isolated influenza HA stem polypeptide, use, kit or composition according to any one of paragraphs 1 to 44, wherein a single dose of isolated influenza HA stem polypeptide is 10 to 30 ug, especially 15 to 25 ug, in particular about 20 ug.

47. The method, emulsion, isolated influenza HA stem polypeptide, use, kit or composition according to any one of paragraphs 1 to 44, wherein a single dose of isolated influenza HA stem polypeptide is 1 to 3 ug, especially 1.5 to 2.5 ug, in particular about 2 ug.

48. The method, emulsion, isolated influenza HA stem polypeptide, use, kit or composition according to any one of paragraphs 1 to 47, wherein the squalene emulsion adjuvant comprises a surfactant selected from poloxamer 401, poloxamer 188, polysorbate 80, sorbitan trioleate, sorbitan monooleate and polyoxyethylene 12 cetyl/stearyl ether either alone, or in combination with each other or in combination with other surfactants.

49. The method, emulsion, isolated influenza HA stem polypeptide, use, kit or composition according to any one of paragraphs 1 to 47, wherein the squalene emulsion adjuvant comprises a surfactant and wherein the surfactant comprises polysorbate 80.

50. The method, emulsion, isolated influenza HA stem polypeptide, use, kit or composition according to paragraph 49, wherein the surfactant consists of polysorbate 80.

51. The method, emulsion, isolated influenza HA stem polypeptide, use, kit or composition according to any one of paragraphs 48 to 50, wherein the amount of surfactant in a single dose of the squalene emulsion adjuvant is at least 0.4 mg, especially 0.4 to 9.5 mg.

52. The method, emulsion, isolated influenza HA stem polypeptide, use, kit or composition according to any one of paragraphs 1 to 51, wherein the squalene emulsion adjuvant has an average droplet size of 50 to 200 nm, especially 120 nm to 180 nm, in particular 140 nm to 180 nm.

53. The method, emulsion, isolated influenza HA stem polypeptide, use, kit or composition according to any one of paragraphs 1 to 52, wherein the squalene emulsion adjuvant has a polydispersity of 0.5 or less, especially 0.3 or less, such as 0.2 or less.

54. The method, emulsion, isolated influenza HA stem polypeptide, use, kit or composition according to any one of paragraphs 1 to 53, wherein the weight ratio of squalene to surfactant in the squalene emulsion adjuvant is 1.71 to 2.8, especially 2 to 2.4, in particular 2.1 to 2.3, such as 2.2.

55. The method, emulsion, isolated influenza HA stem polypeptide, use, kit or composition according to any one of paragraphs 1 to 54, for administration to a human subject.

56. The method, emulsion, isolated influenza HA stem polypeptide, use, kit or composition according to any one of paragraphs 1 to 55, wherein the squalene emulsion adjuvant consists essentially of squalene, surfactant and water, such as squalene, polysorbate 80 and water.

57. The method, emulsion, isolated influenza HA stem polypeptide, use, kit or composition according to any one of paragraphs 1 to 56, wherein the squalene emulsion adjuvant comprises a tocopherol.

58. The method, emulsion, isolated influenza HA stem polypeptide, use, kit or composition according to paragraph 57, wherein the tocopherol is alpha-tocopherol.

59. The method, emulsion, isolated influenza HA stem polypeptide, use, kit or composition according to paragraph 58, wherein the tocopherol is D/L-alpha-tocopherol.

60. The method, emulsion, isolated influenza HA stem polypeptide, use, kit or composition according to any one of paragraphs 1 to 59, wherein the squalene emulsion adjuvant consists essentially of squalene, tocopherol, surfactant and water, such as squalene, D/L-alpha-tocopherol, polysorbate 80 and water.

61. The method, emulsion, isolated influenza HA stem polypeptide, use, or kit according to any one of paragraphs 1 to 60, wherein the isolated influenza HA stem polypeptide and squalene emulsion adjuvant are administered as a co-formulation.

62. The method, emulsion, isolated influenza HA stem polypeptide, use, kit or composition according to paragraph 61, wherein the volume of a single dose is 0.05 ml to 1 ml, such as 0.1 to 0.6 ml.

63. The method, emulsion, isolated influenza HA stem polypeptide, use, kit or composition according paragraph 61, wherein the volume of a single dose is 0.2 to 0.3 ml, such as 0.25 ml.

64. The method, emulsion, isolated influenza HA stem polypeptide, use, kit or composition according paragraph 61, wherein the volume of a single dose is 0.4 to 0.6 ml, such as 0.5 ml.

65. The method, emulsion, isolated influenza HA stem polypeptide, use, kit or composition according to any one of paragraphs 61 to 64, for intramuscular administration.

66. The method, emulsion, isolated influenza HA stem polypeptide, use, or kit according to any one of paragraphs 1 to 8 or 11 to 60 or 62 to 65, wherein the isolated influenza HA stem polypeptide and squalene emulsion adjuvant are administered as separate formulations.

67. The method, emulsion, isolated influenza HA stem polypeptide, use, kit or composition according to paragraph 66, wherein the squalene emulsion adjuvant is for intramuscular administration.

68. The method, emulsion, isolated influenza HA stem polypeptide, use, kit or composition according to paragraph 66, wherein the isolated influenza HA stem polypeptide is for intramuscular administration.

69. The method, emulsion, isolated influenza HA stem polypeptide, use, kit according to any one of paragraphs 66 to 68, wherein the isolated influenza HA stem polypeptide and squalene emulsion adjuvant are administered to a location draining to the same lymph node, such as to the same limb, in particular to the same muscle.

70. The method, emulsion, isolated influenza HA stem polypeptide, use, kit according to any one of paragraphs 66 to 69, wherein the isolated influenza HA stem polypeptide and squalene emulsion adjuvant are administered to the same location.

71. The method, emulsion, isolated influenza HA stem polypeptide, use, kit according to any one of paragraphs 66 to 70, wherein the isolated influenza HA stem polypeptide and squalene emulsion adjuvant are administered concurrently.

72. The method, emulsion, isolated influenza HA stem polypeptide, use, kit or composition according to any one of paragraphs 1 to 71, wherein administration of the isolated influenza HA stem polypeptide and squalene emulsion adjuvant induces an immune response that is at least 2-fold, such as at least 5-fold, such as at least 10-fold, such as at least 100-fold greater than that of the isolated influenza HA stem polypeptide administered without squalene emulsion adjuvant.

73. The method, emulsion, isolated influenza HA stem polypeptide, use, kit or composition according to any one of paragraphs 1 to 72, for administration to a subject which is not infected with influenza virus.

74. The method, emulsion, isolated influenza HA stem polypeptide, use, kit or composition according to any one of paragraphs 1 to 73, wherein the elicited immune response reduces partially or completely the severity of one or more symptoms and/or time over which one or more symptoms of influenza virus infection are experienced by the subject.

75. The method, emulsion, isolated influenza HA stem polypeptide, use, kit or composition according to any one of paragraphs 1 to 74, wherein the elicited immune response reduces the likelihood of developing an established influenza virus infection after challenge.

76. The method, emulsion, isolated influenza HA stem polypeptide, use, kit or composition according to any one of paragraphs 1 to 75, wherein the elicited immune response slows progression of influenza.

### EXAMPLES

### Example 1—Mouse Immunisation

The HA stem-ferritin was generated by expressing the ectodomain of HA fused to H. pylori ferritin with a Ser-Gly-Gly linker, or to the light or the heavy chain of Trichoplusia ni insect ferritin with a Gly-Gly-Ser-Gly-Gly (SEQ ID NO: 12) linker.

### Study A

The immunogenicity of a HA stem H1 candidate vaccine was evaluated in CB6F1 mice. Ten female CB6F1 mice were immunized at days 0 and 28 with:
(a) recombinant HA stem H1ssF A/New Caledonia/20/99 (a polypeptide comprising a H1ssF A/New Caledonia/20/99 stem polypeptide and H. pylori ferritin linked with a Ser-Gly-Gly linker (SEQ ID NO: 6, referred to as ‘recombinant stem’, ‘rec’ ‘rec stem’ and ‘protein’ in the figures)) without adjuvant,
(b) recombinant HA stem H1ssF A/New Caledonia/20/99 (a polypeptide comprising a H1ssF A/New Caledonia/20/99 stem polypeptide and H. pylori ferritin linked with a Ser-Gly-Gly linker (SEQ ID NO: 6)) adjuvanted with 25 uL AS03,
(c) QIV (commercially available quadrivalent influenza vaccine comprising inactivated split influenza virions of the strains A/Brisbane/02/2018 H1N1pdm09, A/Kansas/14/2017 H3N2, B/Colorado/06/2017 (B/Victoria) and B/Phuket/3073/2013 (B/Yamagata)) without adjuvant, (d) QIV formulated with 25 uL AS03, or
(e) NaCl solution.

Serum samples were collected and analysed as described in examples 3 to 7 below using the assay protocols described in example 2.

Non-inferiority can be concluded if the lower limit (LL) of the 90% CI for the ratio of the GMTs (GMR) between the compared groups is ≥0.5. Biological/clinical significance (non-inferiority margin) can be concluded if the GMR+90% CI is >0.5. Statistical superiority can be concluded if the GMR+90% CI is ≥2.

### Study B

A further subsequent study, analogous to Study A above, was conducted to investigate the impact of administering different doses of HA stem polypeptide and HA stem polypeptide derived from different strains of influenza. Female CB6F1 mice were immunized with:
(a) recombinant stem H1ssF A/New Caledonia/20/99 (a polypeptide comprising a H1ssF A/New Caledonia/20/99 stem polypeptide and H. pylori ferritin linked with a Ser-Gly-Gly linker (SEQ ID NO: 6)) without adjuvant,
(b) recombinant stem H1ssF A/New Caledonia/20/99 (a polypeptide comprising a H1ssF A/New Caledonia/20/99 stem polypeptide and H. pylori ferritin linked with a Ser-Gly-Gly linker (SEQ ID NO: 6)) adjuvanted with 25 uL AS03,
(c) recombinant stem H1ssF A/Michigan/45/2015 (a polypeptide comprising a H1ssF A/Michigan/45/2015 stem polypeptide and H. pylori ferritin linked with a Ser-Gly-Gly linker (SEQ ID NO: 7)) without adjuvant,
(d) recombinant stem H1ssF A/Michigan/45/2015 (a polypeptide comprising a H1ssF A/Michigan/45/2015 stem polypeptide and H. pylori ferritin linked with a Ser-Gly-Gly linker (SEQ ID NO: 7)) adjuvanted with 25 uL AS03,
(e) recombinant stem H10ssF A/Jiangxi-Donghu/346/2013 (a polypeptide comprising an H10ssF A/Jiangxi-Donghu/346/2013 stem polypeptide and H. pylori ferritin linked with a Ser-Gly-Gly linker, referred to in the figures as ‘H10’ (SEQ ID NO: 9)) without adjuvant,
(f) recombinant stem H10ssF A/Jiangxi-Donghu/346/2013 (a polypeptide comprising an H10ssF A/Jiangxi-Donghu/346/2013 stem polypeptide and H. pylori ferritin linked with a Ser-Gly-Gly linker, referred to in the figures as ‘H10’ (SEQ ID NO: 9)) adjuvanted with 25 uL AS03, (g) QIV without adjuvant,
(h) QIV formulated with 25 uL AS03, or
(i) NaCl solution.

Fourteen mice were included per group (a)-(h) and four mice were included in group (i). Serum samples were collected and analysed as described in examples 3 to 7 below using the assay protocols described in example 2.

Non-inferiority can be concluded if the lower limit (LL) of the 90% CI for the ratio of the GMTs (GMR) between the compared groups is >0.5. Biological/clinical significance (non-inferiority margin) can be concluded if the GMR+90% CI is >0.5. Statistical superiority can be concluded if the GMR+90% CI is ≥2.

### Study C

Another study was conducted to investigate the immunogenicity of HA stem from H1 and/or H10 with insect ferritin and to assess non-inferiority for the heterotypic (insect) compared to the homotypic (H. pylori) ferritin constructs.

The immunogenicity of a recombinant HA stem H1 candidate vaccine, a recombinant HA stem H10 candidate vaccine and a recombinant HA stem H1+H10 vaccine, was evaluated in CB6F1 mice. Twenty female CB6F1 mice were immunized at days 0 and 28 with:
(a) recombinant stem H1ssF A/Michigan/45/2015 (a polypeptide comprising a H1ssF A/Michigan/45/2015 25 stem polypeptide and H. pylori ferritin linked with a Ser-Gly-Gly linker (SEQ ID NO: 7, referred to as ‘recombinant stem’, ‘rec’ ‘rec stem’ and ‘protein’ in the figures) adjuvanted with 25 uL AS03,
(b) recombinant stem H10 A/Jiangxi Donghu/346/2013 (a polypeptide comprising a H10 A/Jiangxi Donghu/346/2013 stem polypeptide and H. pylori ferritin linked with a Ser-Gly-Gly linker (SEQ ID NO: 9), referred to as ‘recombinant HA stem’ in the figures) adjuvanted with 25 uL AS03,
(c) recombinant stem H1ssF A/Michigan/45/2015 and H. pylori ferritin (SEQ ID NO: 7) mixed with recombinant stem H10 A/Jiangxi Donghu/346/2013 and H. pylori ferritin (SEQ ID NO: 9) adjuvanted with 25 uL AS03,
(d) recombinant stem H1ssF A/Michigan/45/2015 stem polypeptide with recombinant stem H10 A/Jiangxi Donghu/346/2013 stem polypeptide and insect ferritin (SEQ ID NO: 15) adjuvanted with 25 uL AS03.

Eight female CB6F1 mice were immunized at days 0 and 28 with:
(e) QIV (commercially available quadrivalent influenza vaccine comprising inactivated split influenza virions of the strains A/Brisbane/02/2018 H1N1pdm09, A/Kansas/14/2017 H3N2, B/Colorado/06/2017 (B/Victoria) and B/Phuket/3073/2013 (B/Yamagata)) without adjuvant,
(f) QIV formulated with 25 uL AS03 and four mice with (g) NaCl solution.

Serum samples were collected and analysed as described in examples 3 to 7 below using the assay protocols described in example 2.

Non-inferiority can be concluded if the lower limit (LL) of the 90% CI for the ratio of the GMTs (GMR) between the compared groups is >0.5. Statistical superiority can be concluded if the LL of the 90% CI for the ratio of the GMTs (GMR) between the compared groups is >1.

### Example 2—Assay Protocols

### Anti-HA IgG Antibodies by ELISA

Quantification of mouse anti-HA IgG antibodies was performed by ELISA using HA antigen (full length or stem only) as coating diluted at a concentration of 4 μg/ml in PBS (50 μl/well). The plates were then incubated for 1 hour at 37° C. in saturation buffer. Diluted sera were added to the coated plates (50 μl/well) and incubated for 90 minutes at 37° C. The plates were washed prior to the adding of diluted peroxydase conjugated goat anti-mouse IgG. The reaction was stopped with H2SO4 2N and optical densities were read at 490-620 nm. The titers were expressed as ELISA Units Titers (EU/ml).

### Stem Specific T Cell Frequencies

Spleens were collected and placed in complemented RPMI Cell suspensions were prepared from each spleen using a tissue grinder. The splenic cell suspensions were filtered, harvested, centrifuged and resuspended in Complete Medium. Fresh splenocytes were then plated in 96-well plates in presence of overlapping peptide pool covering the sequence of H1 Mich 15 stem. Following stimulation, cells were stained and analyzed using a 5-colour ICS assay. Cells were washed and stained with anti-CD16/32, anti-CD4-V450 and anti-CD8-PerCp-Cy5.5 antibodies. Live/dead-PO was added for 30 min at 4° C. Cells were permeabilized and stained with anti-IL2-FITC, anti-IFNγ-APC and anti-TNFα-PE antibodies. Stained cells were analyzed by flow cytometry using a LSRII and the FlowJo software.

### Neutralization Antibody Titers

Quantification of mouse neutralizing antibody titers was assessed by microneutralization assay. Briefly, mouse sera were diluted and incubated in presence of reporter influenza virus. After incubation, the serum-virus mix were added on cell culture. Influenza-positive cells were analysed and quantified by flow cytometry. Titers are expressed as 50% neutralization titers (IC50), corresponding to reduction titers calculated by regression analysis of the inverse dilution of serum that provided 50% cell infected reduction compared to control wells (virus only, no serum).

### Example 3—Anti-H1 Stem IgG Antibody Titers by ELISA at 14 Days Post Dose 2

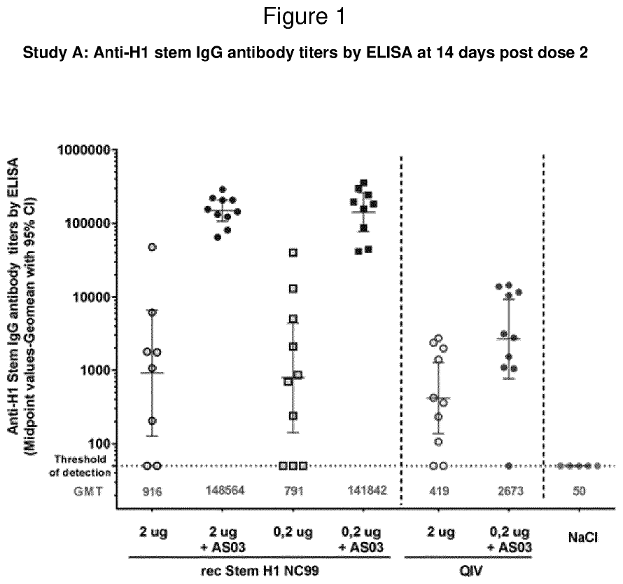

IgG antibody titers directed towards H1-stem were measured by ELISA assay using a stem construct at 14 days post second immunization (day 42). The results from Study A are shown in FIG. 1 ELISA titers are expressed as midpoint values (Geomean with 95% Cl). High anti-H1 stem IgG antibodies are induced by H1 stem recombinant antigen adjuvanted with AS03 compared to the non-adjuvanted H1 stem recombinant antigen: increase is from 179-fold to 162-fold for the 0.2 ug and 2 μg antigen dose respectively. The non-inferiority of H1 stem recombinant antigen adjuvanted with AS03 over non-adjuvanted QIV and QIV-AS03 was shown (LL of 90% CI of GMR is above 0.5-fold). The data even suggest that this H1 stem recombinant antigen adjuvanted with AS03 induced higher responses than non-adjuvanted QIV and QIV-AS03 (increase is close to 338-fold and 53-fold respectively).

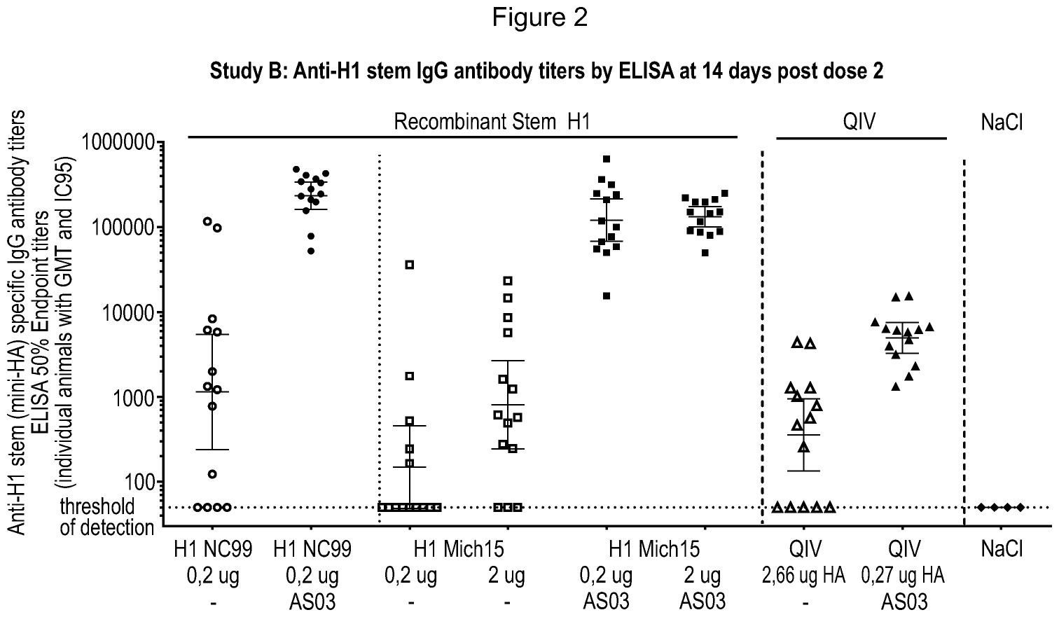

Results from Study B are shown in FIG. 2. ELISA titers are expressed as 50% endpoint titers (individual animals with GMT and IC95). High anti-H1 stem IgG antibodies are induced by both H1 stem NC99 and Mi15 recombinant antigen (named as H1ssF NC99 and H1ssF Mi15) adjuvanted with AS03 compared to the non-adjuvanted H1 stem recombinant antigens: increase is from 164-fold to 813-fold. H1 stem recombinant antigens adjuvanted with AS03 induced higher anti-H1 stem IgG antibodies than non-adjuvanted QIV (340-fold higher for H1ssF Mi15 and 659-fold higher for H1ssF NC99) and QIV-AS03 (24-fold higher for H1ssF Mi15 and 47-fold higher for H1ssF NC99).

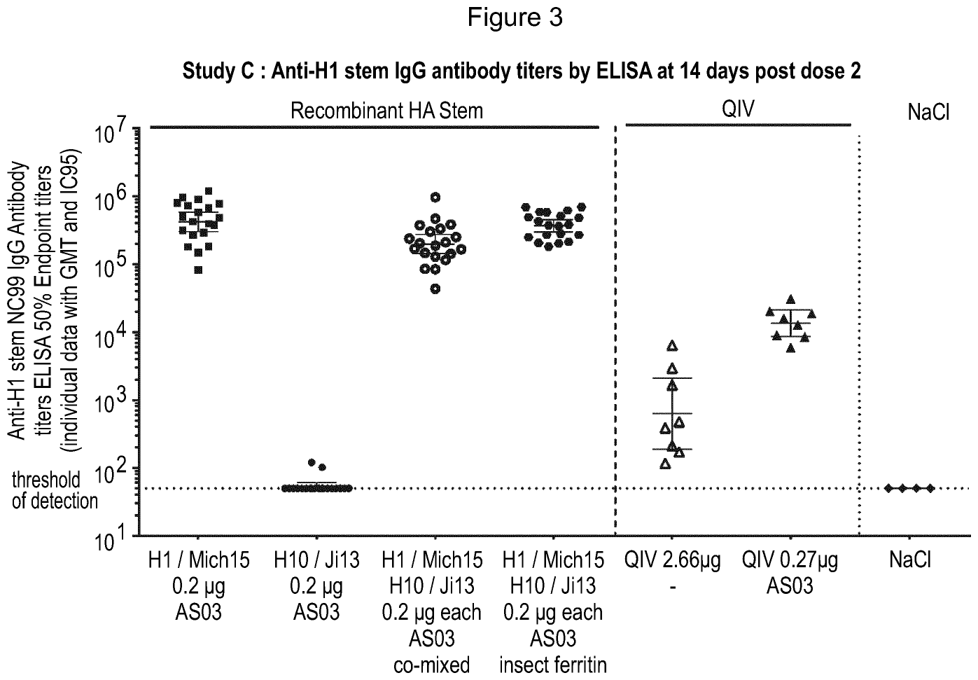

The results from Study C are shown in FIG. 3. ELISA titers are expressed as endpoint values (individual data with GMT and IC95). The non-inferiority of recombinant H1/H10 stem heterotypic construct (insect ferritin) over recombinant H1/Mich15 homotypic construct (H. pylori ferritin) was shown (LL of 90% CI of GMR above 0.5-fold). The data even suggest that this heterotypic construct induced higher responses than H1/Mich15 homotypic construct co-mixed with H10/Ji13 homotypic construct: increase is closed to 2-fold (estimated GMR of 1.86 with 95% CI from 1.39-fold to 2.49-fold) The recombinant H1/Mich15 homotypic construct (H. pylori ferritin) co-mixed with the recombinant the recombinant H10/Ji13 homotypic construct (H. pylori ferritin) induced lower anti-H1 stem IgG antibody titers compared to Recombinant H1/Mich15 homotypic construct (H. pylori ferritin) (non-inferiority not demonstrated, LL of 90% Cl of GMR below 0.5-fold).

The dotted horizontal line on the figures corresponds to the threshold of detection.

### Example 4—Anti-H1/NC/99 and Anti-H1/Mich/15 IgG Antibody Titers by ELISA at 14 Days Post Dose 2

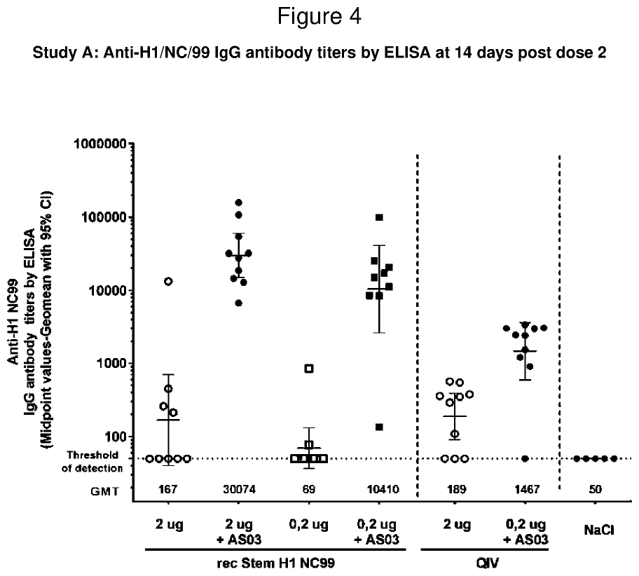

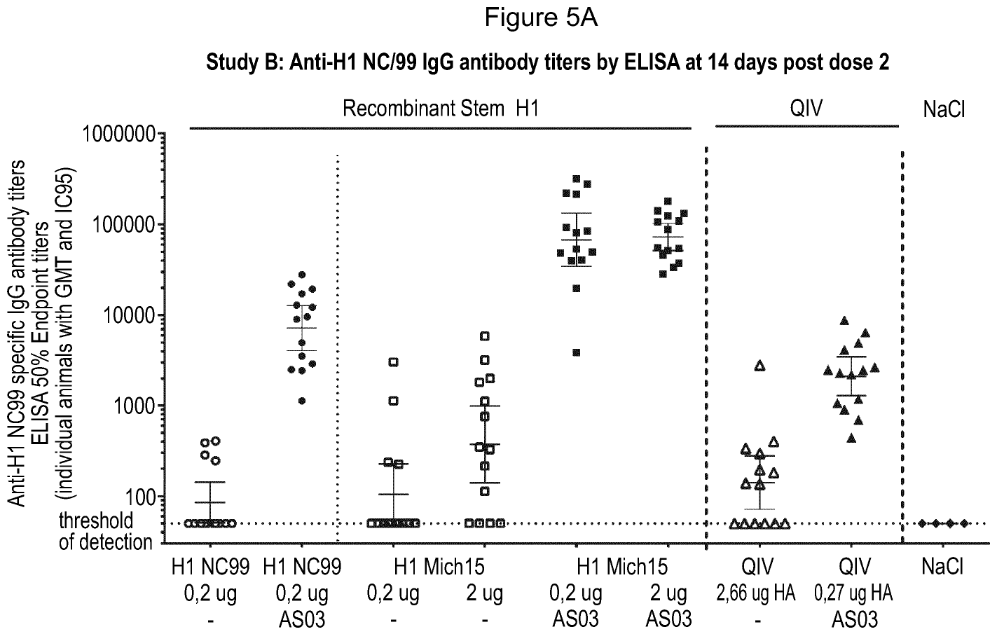

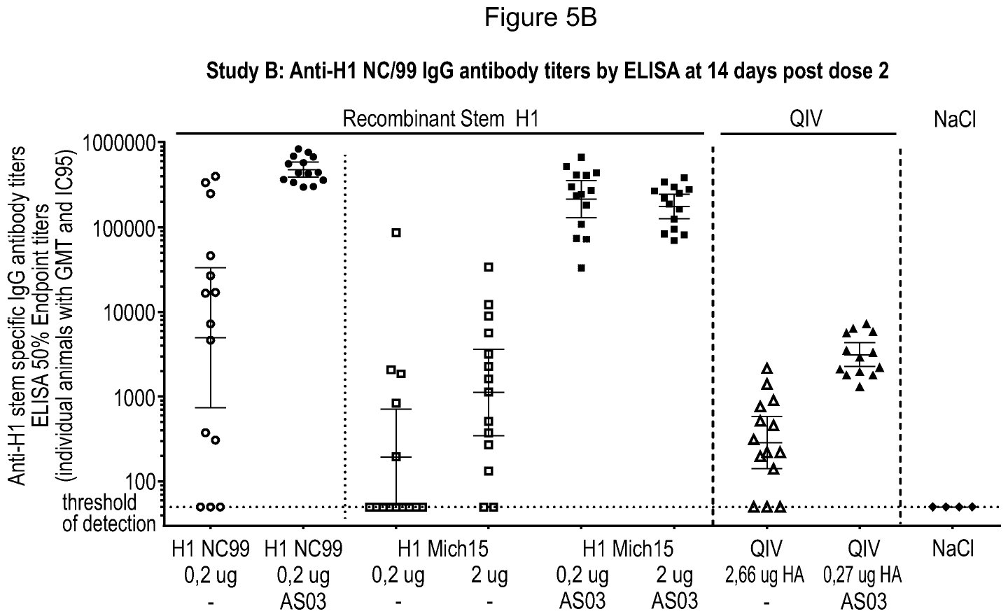

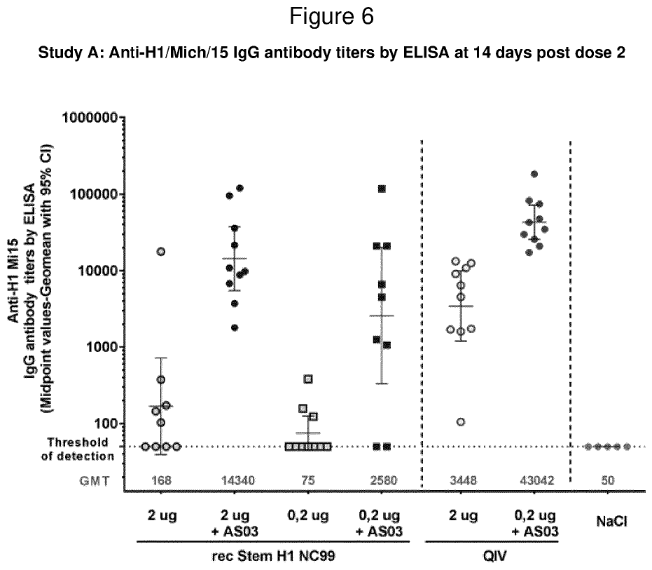

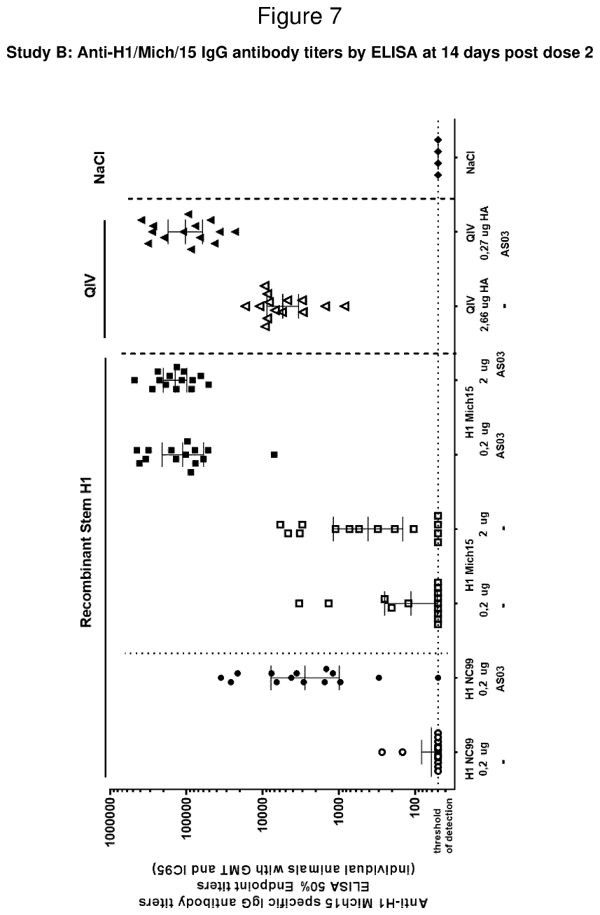

IgG antibody titers directed towards H1 were measured by ELISA assay using a full-length A/H1N1/New Caledonia/20/1999 polypeptide (trimeric protein with foldon and without transmembrane domain) (Study A, FIG. 4 and Study B, FIG. 5A) or a full-length A/H1N1/Michigan/2015 polypeptide (trimeric protein with foldon and without transmembrane domain) (Study A, FIG. 6 and Study B, FIG. 7) at 14 days post second immunization (day 42). High anti-H1NC99 and anti-H1 Mich15 IgG antibodies were induced by recombinant H1 stem antigen adjuvanted with AS03 compared to the non-adjuvanted H1 stem recombinant antigen: increase is from 34-fold to 180-fold for the 0.2 ug and 2 μg antigen dose respectively (Study A). The non-inferiority of H1 stem recombinant antigen adjuvanted with AS03 over non-adjuvanted QIV and QIV-AS03 was shown for anti-H1NC99 IgG antibody response (LL of 90% CI of GMR is above 0.5-fold) (Study A). The data even suggest that this H1 stem recombinant antigen adjuvanted with AS03 induced higher responses than non-adjuvanted QIV (increase is close to 55-fold with LL of the 90% CI=12.6 and UL of the 90% CI=240.5) and QIV-AS03 (increase is close to 7-fold with LL of the 90% CI=1.52 and UL of the 90% CI=33.10) (Study A).

In Study B only, the experiment was repeated using a stem-only A/H1N1/New Caledonia/20/1999 polypeptide as coating antigen. The results are shown in FIG. 5B. High anti-H1NC99 and anti-H1 Mich15 IgG antibodies are induced by both H1 stem NC99 and Mi15 recombinant antigen (named as H1ssF NC99 and H1ssF Mi15) adjuvanted with AS03 compared to the non-adjuvanted H1 stem recombinant antigens: increase is from 46-fold to 992. H1 stem recombinant antigens adjuvanted with AS03 induced higher anti-H1NC99 IgG antibodies than non-adjuvanted QIV (480-fold higher for H1ssF Mi15-AS03 and 51-fold higher for H1ssF NC99-AS03) and QIV-AS03 (32-fold higher for H1ssF Mi15-AS03 and 3.4-fold higher for H1ssF NC99-AS03).

For FIG. 4 and FIGS. 6A and 6B, ELISA titers are expressed as midpoint values (Geomean with 95% CI). For FIGS. 5A and 5B and FIG. 7, ELISA titers are expressed as 50% endpoint titers (individual animals with GMT and IC95).

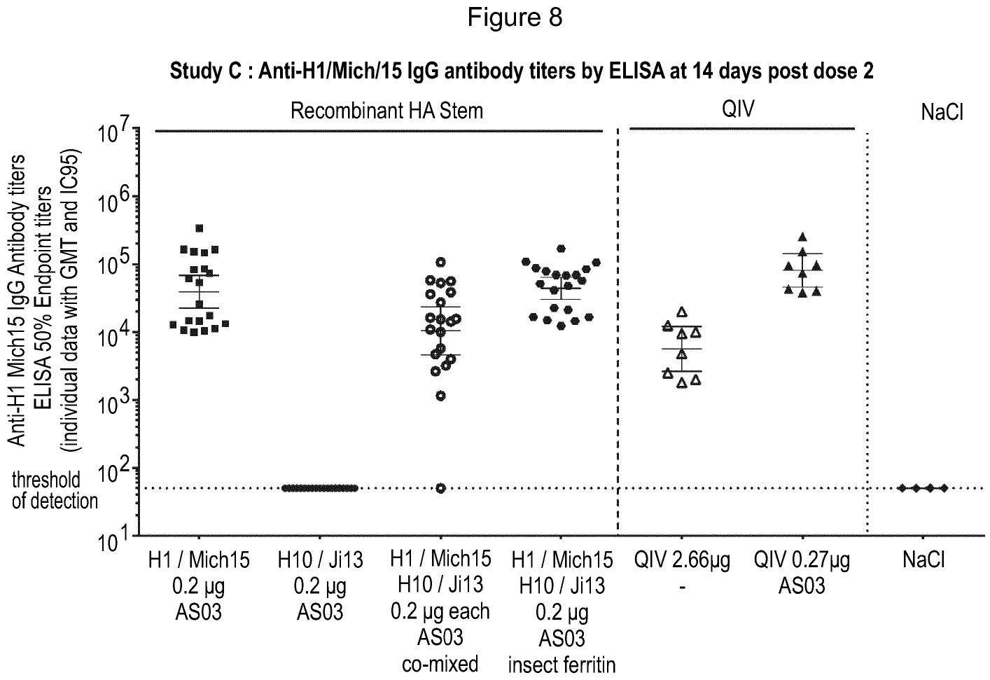

The results from Study C are shown in FIG. 8. IgG antibody titers directed towards H1 were measured by ELISA assay using a full-length (trimeric protein with foldon and without transmembrane domain) A/H1N1/Michigan/2015 polypeptide at 14 days post second immunization (day 42). ELISA titers are expressed as endpoint values. The non-inferiority of Recombinant H1/H10 stem heterotypic construct (insect ferritin) over Recombinant H1/Mich15 homotypic construct (H. pylori ferritin) was shown (LL of 90% CI of GMR above 0.5-fold). The recombinant H1/Mich15 homotypic construct (H. pylori ferritin) co-mixed with the recombinant the recombinant H10/Ji13 homotypic construct (H. pylori ferritin) induced lower anti-H1/Mich/15 IgG antibody titers compared to Recombinant H1/Mich15 homotypic construct (H. pylori ferritin) (non-inferiority not demonstrated, LL of 90% CI of GMR below 0.5-fold).

The dotted horizontal line on the figures corresponds to the threshold of detection.

### Example 5—Anti-Group A1 (H2, H9, H18) IgG Antibody Titers by ELISA at 14 Days Post Dose 2

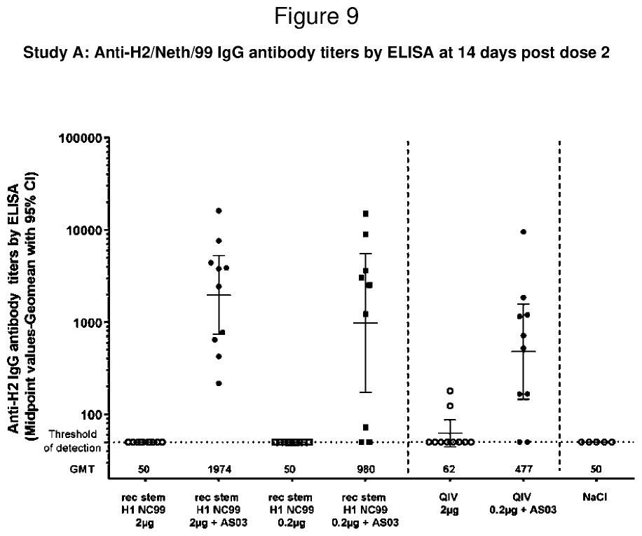

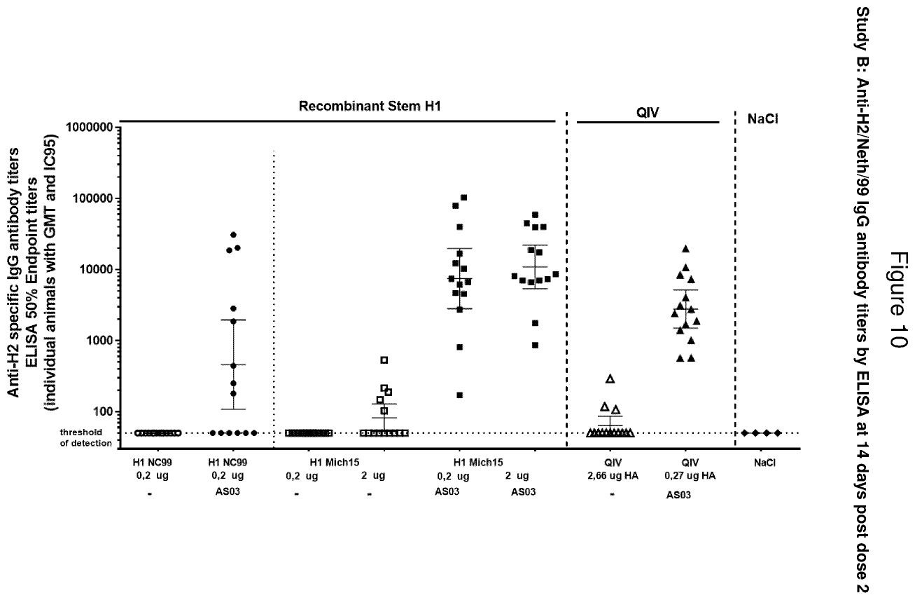

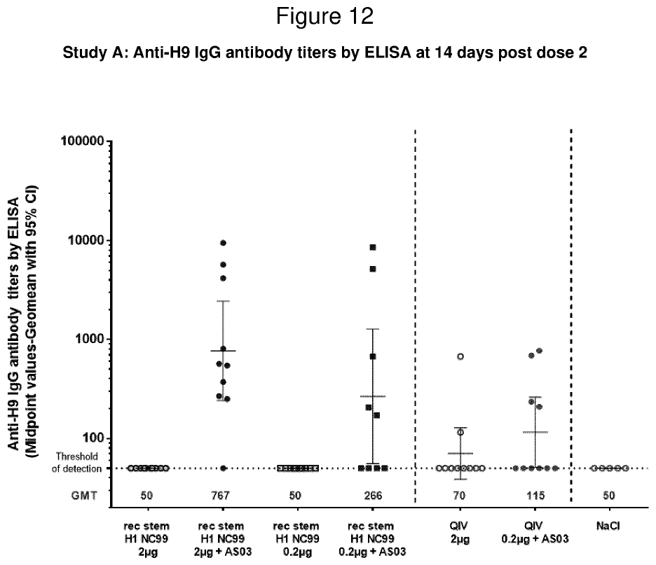

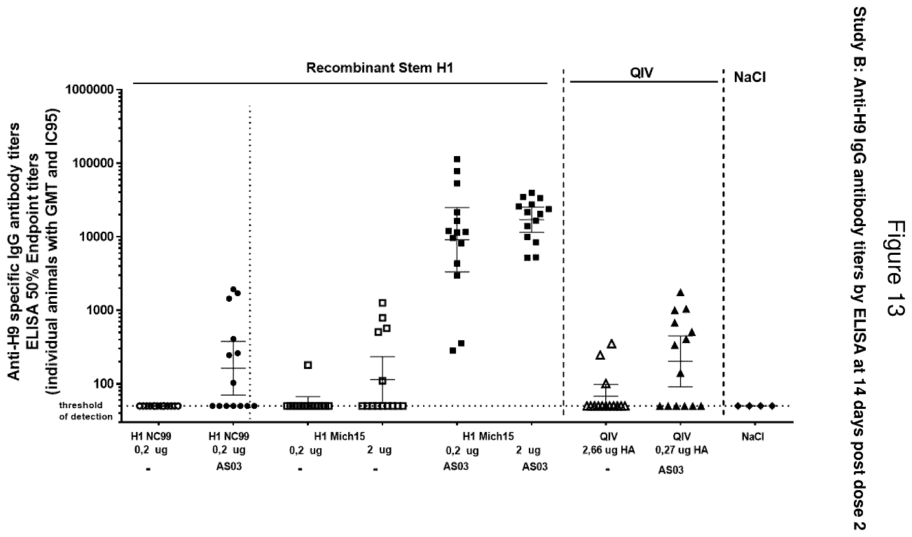

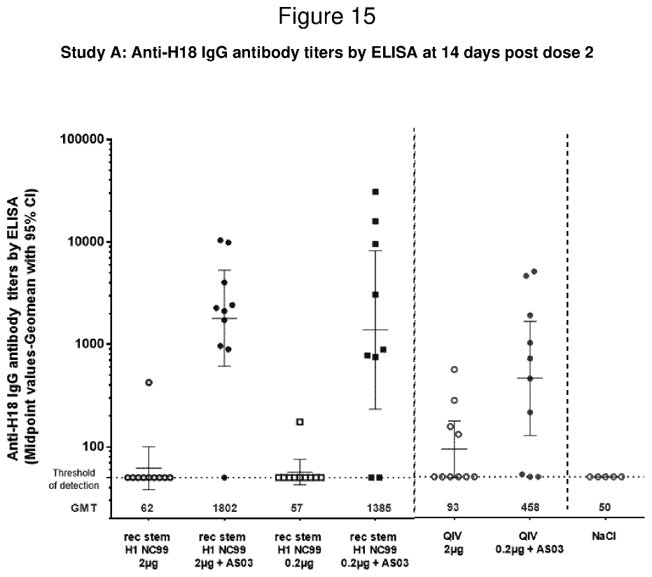

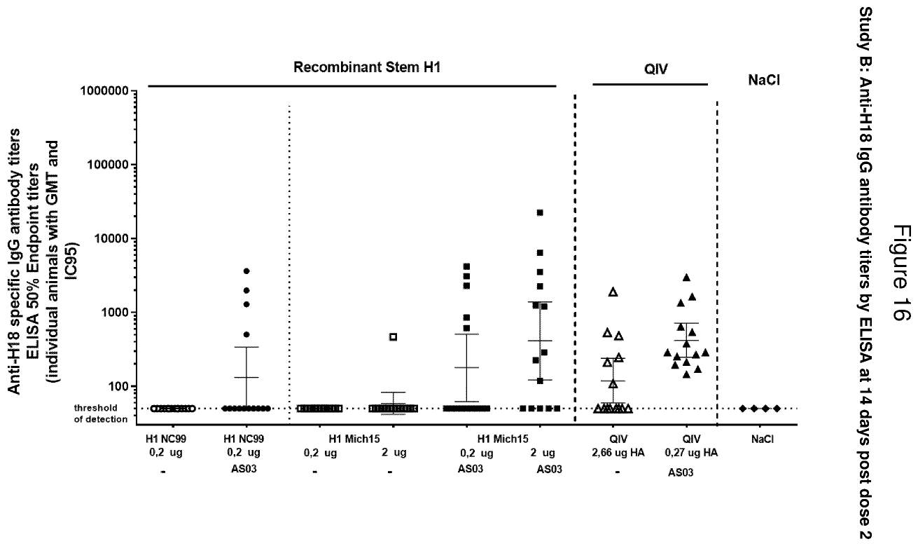

IgG antibody titers directed towards group A1 HA were measured by ELISA assay using a full-length H2 protein (Study A, FIG. 9 and Study B, FIG. 10), a full-length H9 protein (Study A, FIG. 12 and Study B, FIG. 13) or a full length H18 protein (Study A, FIG. 15 and Study B, FIG. 16) at 14 days post second immunization (day 42). High anti-H2, anti-H9 and anti-H18 IgG antibodies were induced by recombinant H1 stem antigen adjuvanted with AS03. compared to the non-adjuvanted H1 stem recombinant antigen: increase is 39-fold and 20-fold for the 2 ug and 0.2 μg antigen dose respectively for anti-H2, 15-fold and 5-fold for the 2 ug and 0.2 μg antigen dose respectively for anti-H9 and 29-fold and 24-fold for the 2 ug and 0.2 μg antigen dose respectively (Study A).

For Study A FIGS. 9, 12 and 15, ELISA titers are expressed as midpoint values (Geomean with 95% Cl). For Study B FIGS. 10, 13 and 15, ELISA titers are expressed as 50% endpoint titers (individual animals with GMT and IC95).

High anti-H2, anti-H9 and anti-H18 IgG antibodies are induced by H1 stem Mi15 recombinant antigen (named H1ssF Mi15) adjuvanted with AS03 compared to the non-adjuvanted H1 stem recombinant antigen: increase is from 2.63-fold to 166. H1 stem Mi15 recombinant antigens adjuvanted with AS03 induced higher anti-H2 and anti-H9 IgG antibodies than non-adjuvanted QIV (116-fold higher for anti_H2 IgG response and 134-fold higher for anti-H9 IgG response) and QIV-AS03 (2.7-fold higher for anti_H2 IgG response and 45-fold higher for anti-H9 IgG response).

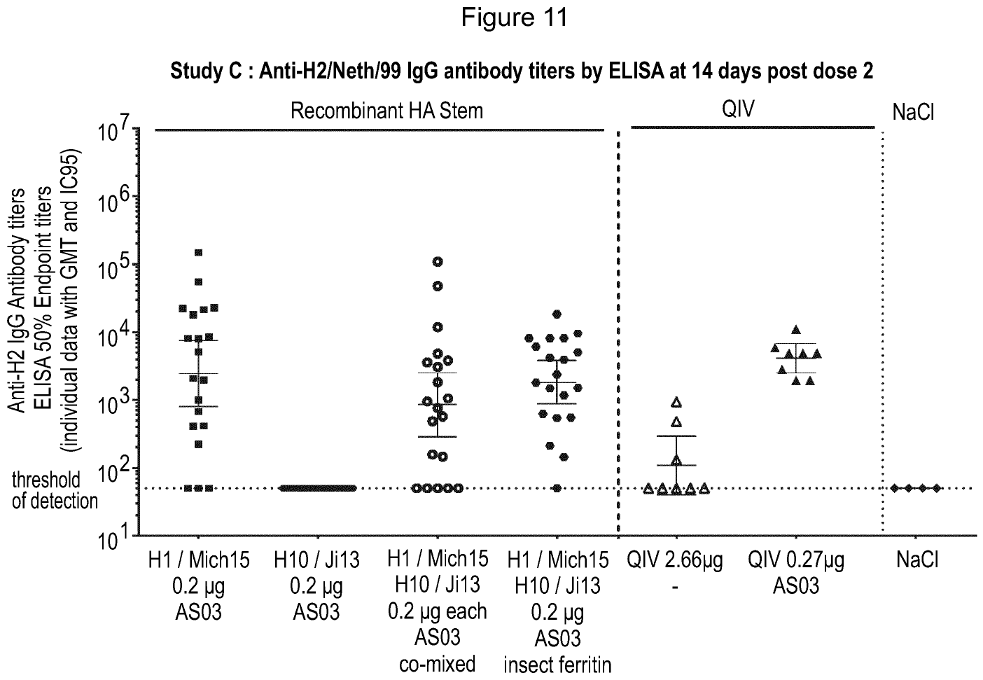

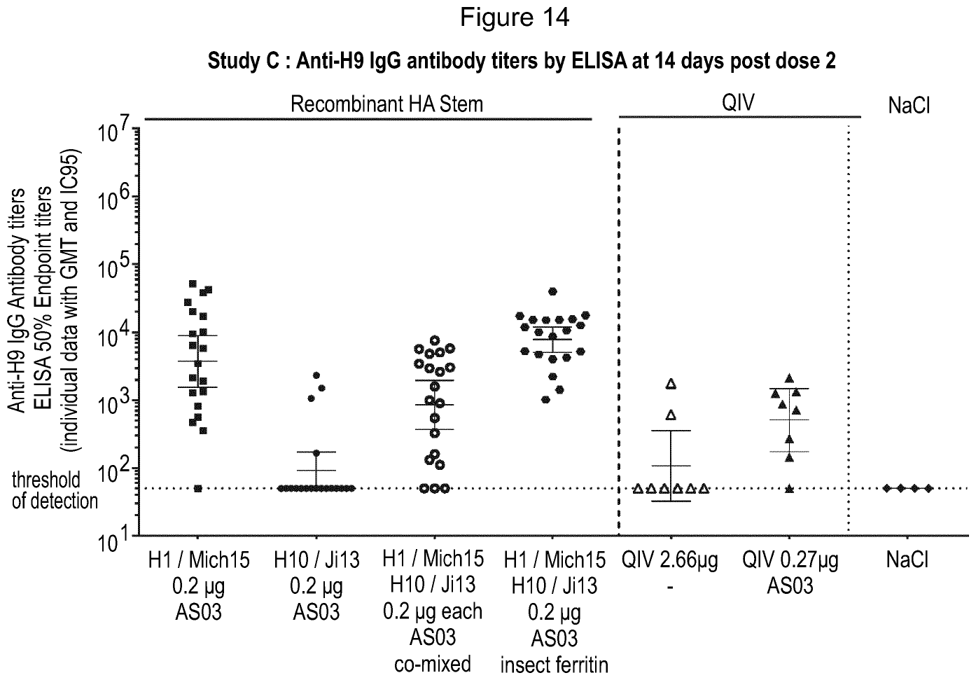

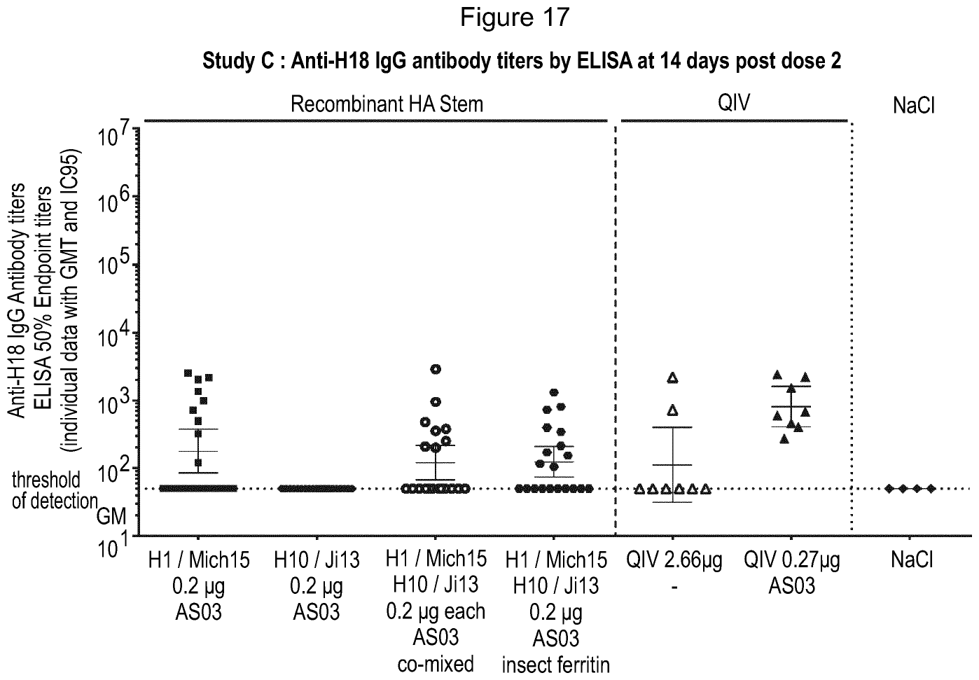

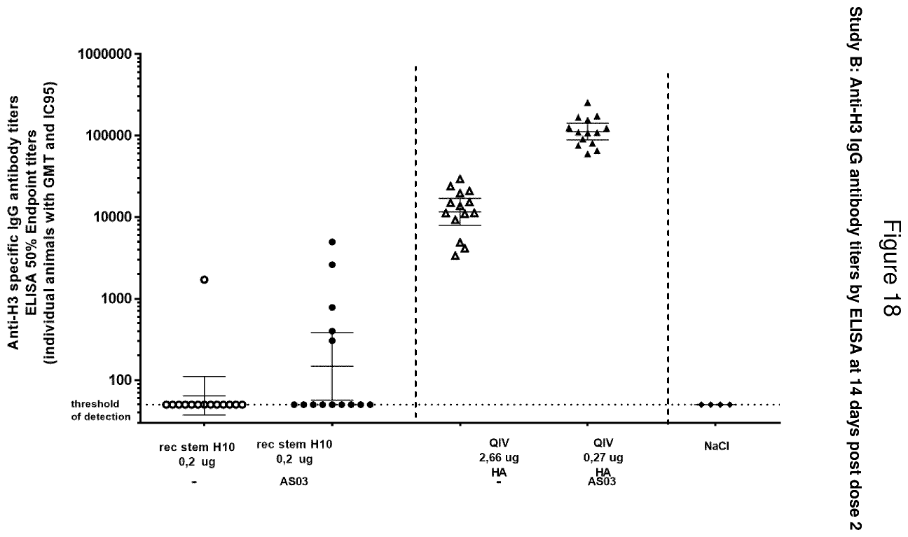

In Study C, IgG antibody titers directed towards group A1 HA measured by ELISA assay using a full-length H2 (FIG. 11), a full-length H9 (FIG. 14) or a full-length H18 HA stem (FIG. 17) were measured at 14 days post second immunization (day 42). ELISA titers are expressed as endpoint values (individual values with GMT and 95% CI). The non-inferiority of Recombinant H1/H10 stem heterotypic construct (insect ferritin) over Recombinant H1/Mich15 homotypic construct (H. pylori ferritin) was shown (LL of 90% CI of GMR above 0.5-fold) for anti-H9 IgG antibodies. Lower anti-H2 and anti-H18 IgG antibodies are induced by the Recombinant H1/H10 stem heterotypic construct (insect ferritin) compared to the Recombinant H10 stem homotypic construct (H. pylori ferritin). The recombinant H1/Mich15 homotypic construct (H. pylori ferritin) co-mixed with the recombinant the recombinant H10/Ji13 homotypic construct (H. pylori ferritin) induced lower anti-H2, H9 and H18 IgG antibody titers compared to Recombinant H1/Mich15 homotypic construct (H. pylori ferritin) (non-inferiority not demonstrated, LL of 90% CI of GMR below 0.5-fold).

The dotted horizontal line on the figures corresponds to the threshold of detection.

### Example 6—Anti-Group A2 (H3, H7, H10) IgG Antibody Titers by ELISA at 14 Days Post Dose 2

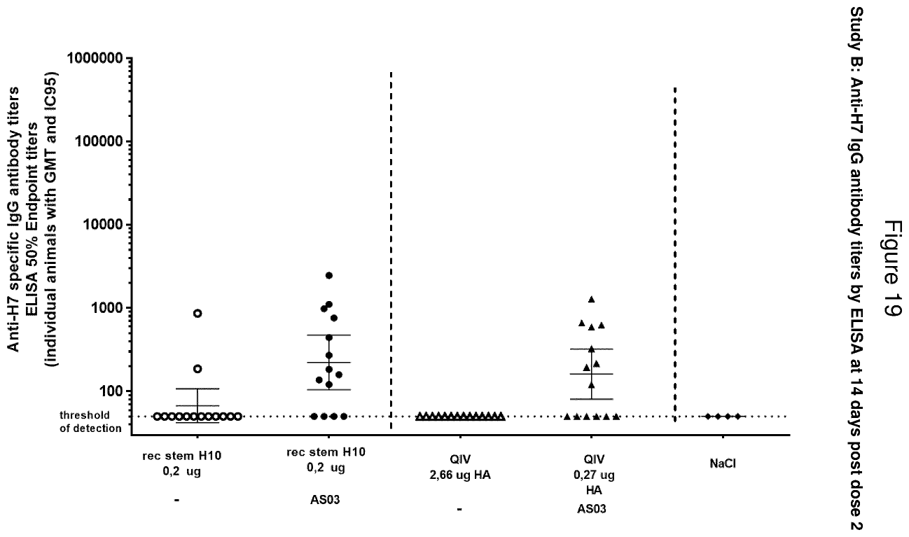

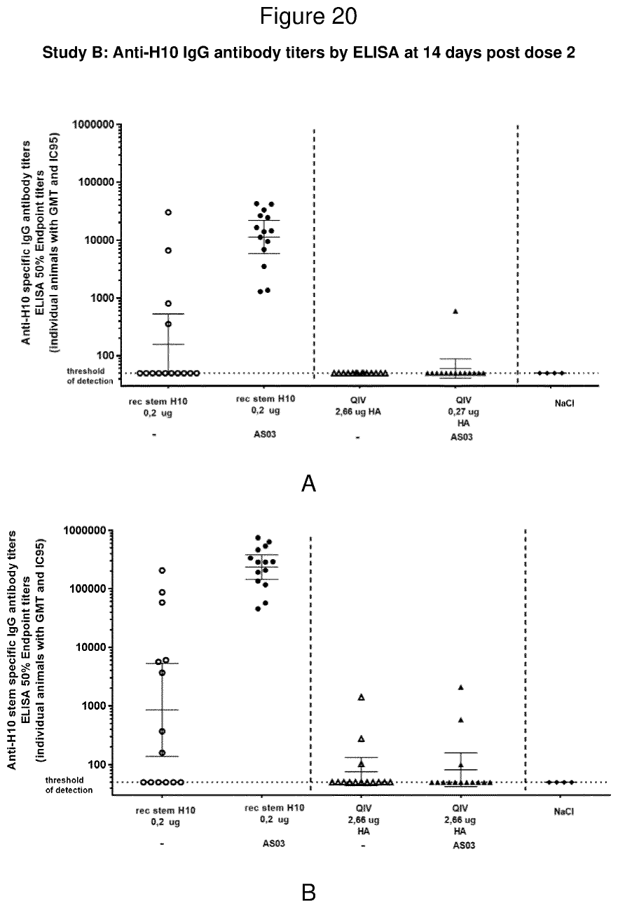

This experiment was carried out for Study B. IgG antibody titers directed towards group A2 HA were measured by ELISA assay using full-length H3, protein (FIG. 18), a full-length H7 protein (FIG. 19) or a full length H10 protein (FIG. 20A) at 14 days post second immunization (day 42). ELISA titers are expressed as midpoint values. Anti-H3, anti-H7 and anti-H10 IgG antibodies were induced by recombinant H10 stem antigen adjuvanted with AS03.

This Study B experiment was repeated using a stem-only H10 stem polypeptide as coating antigen. The results are shown in FIG. 20B. High anti-H10 IgG antibodies were induced by H10 Ji13 stem recombinant antigen (named H10ssF Ji13) adjuvanted with AS03 compared to the non-adjuvanted H10 Ji13 stem recombinant antigen (72-fold higher). Anti-H3 and anti-H7 IgG antibodies were induced by H10 Ji13 stem recombinant antigen adjuvanted with AS03.

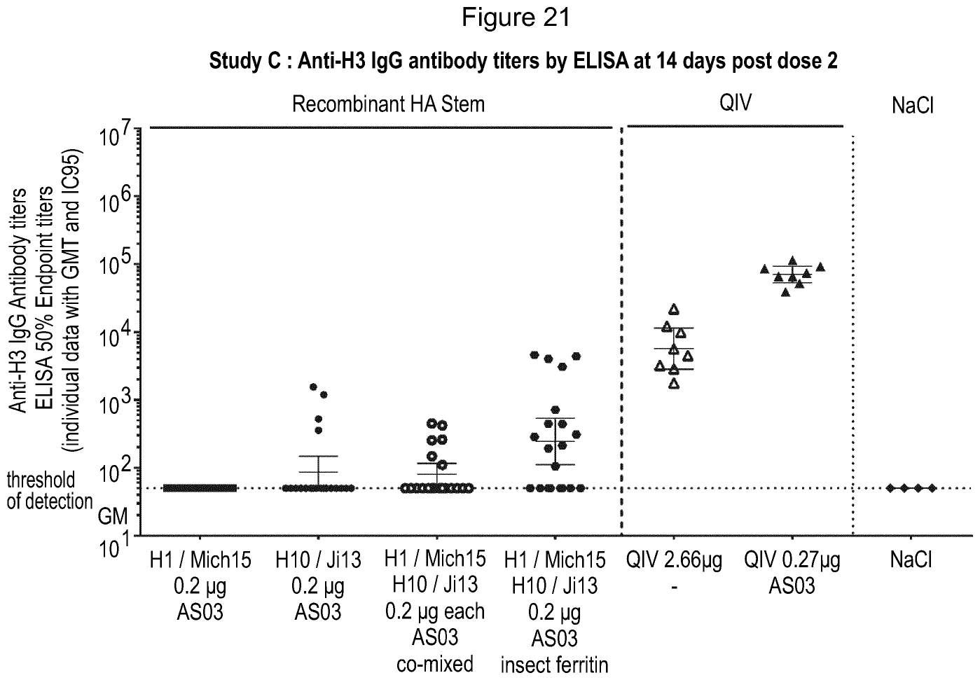

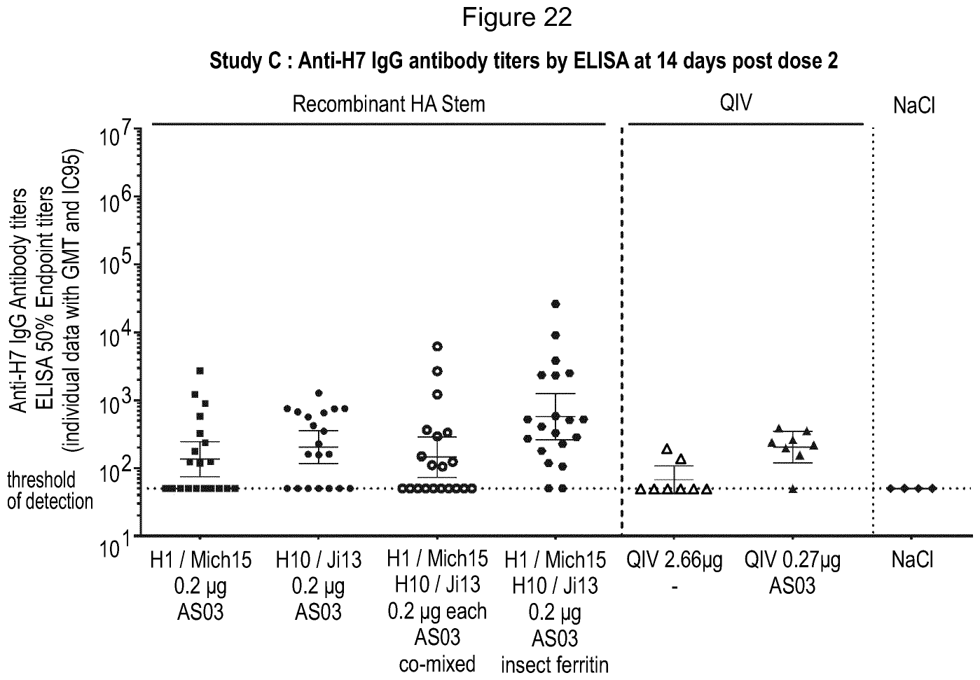

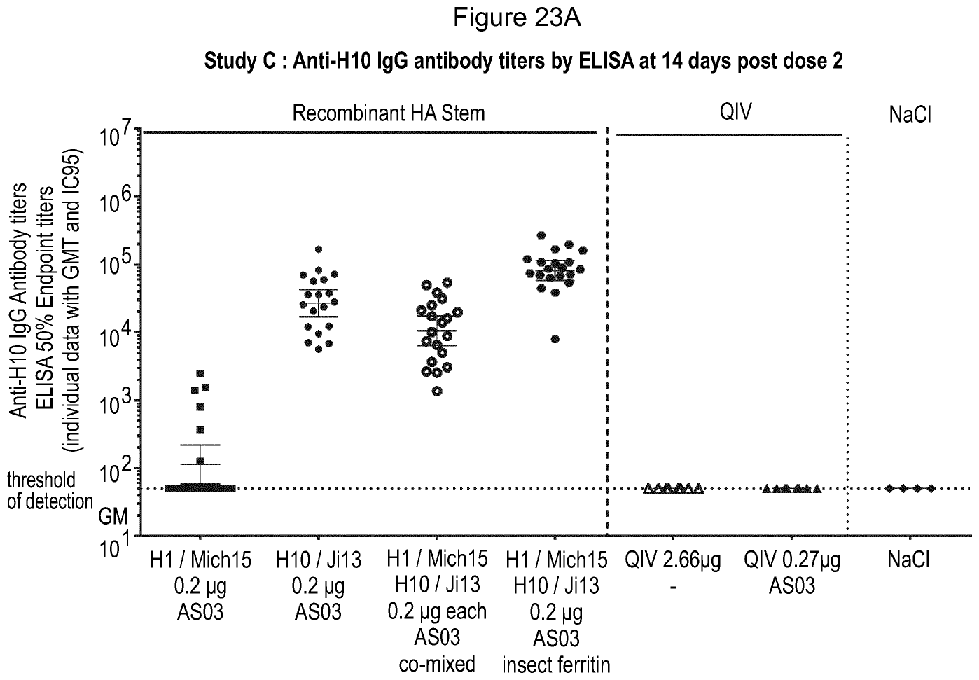

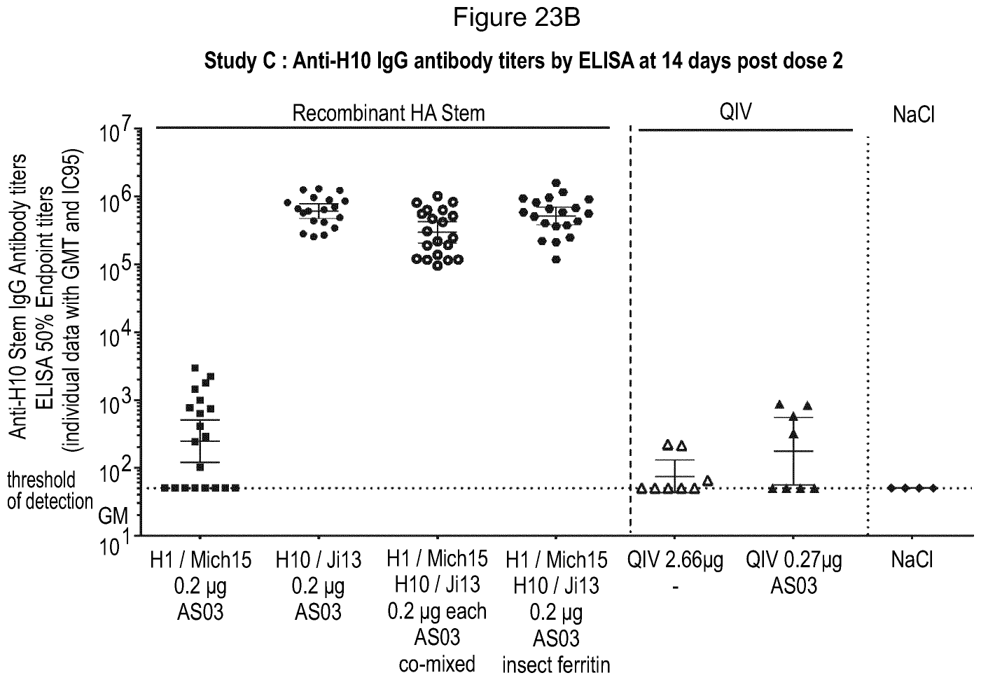

This experiment was further carried out for Study C and results are shown in FIGS. 21, 22 and 23. IgG antibody titers directed towards group A2 HA were measured by ELISA assay using full-length H3, protein (FIG. 21), a full-length H7 protein (FIG. 22) or a full length H10 protein (FIG. 23A) at 14 days post second immunization (day 42). ELISA titers are expressed as endpoint values. The non-inferiority of the Recombinant H1/H10 stem heterotypic construct (insect ferritin) over H10/Ji13 homotypic construct (H. pylori ferritin) was shown (LL of 90% CI of GMR above 0.5-fold). The data even suggest that this H1/H10 stem recombinant antigen adjuvanted with AS03 induced higher responses compared to the responses induced by the H10/Ji13 homotypic construct (H. pylori ferritin) (estimated GMR of 2.9 with LL of the 90% CI=1.9 and UL of the 90% CI=4.3 for anti-H10 IgG antibody response) and H1/Mich15 homotypic construct co-mixed with H10/Ji13 homotypic construct (estimated GMR of 7.68 with LL of the 90% CI=4.73 and UL of the 90% CI=12.45 for anti-H10 IgG antibody response). High anti-H3, anti-H7 and anti-H10 IgG antibodies were induced by Recombinant H1/H10 insect ferritin.

This Study C experiment was repeated using a H10 stem polypeptide as coating antigen. The results are shown in FIG. 23B.

The dotted horizontal line on the figures corresponds to the threshold of detection.

### Example 7—H1/Mich/15 Stem Specific CD4+ and CD8+ T Cell Frequencies at 14 Days Post Dose 2

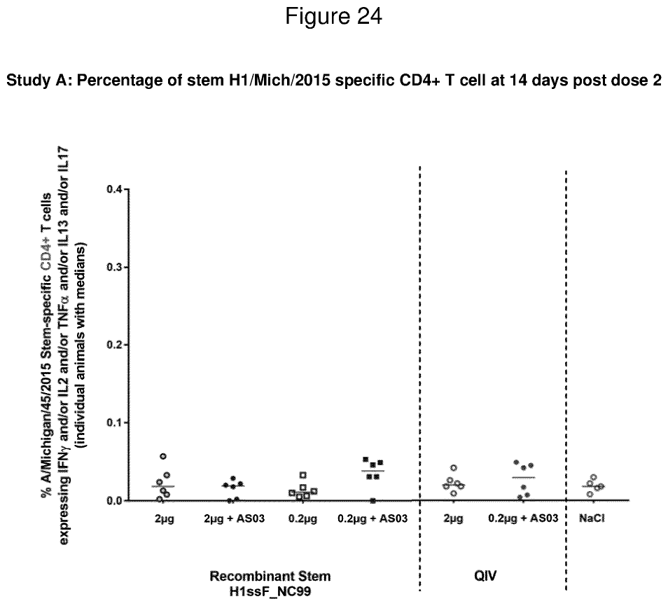

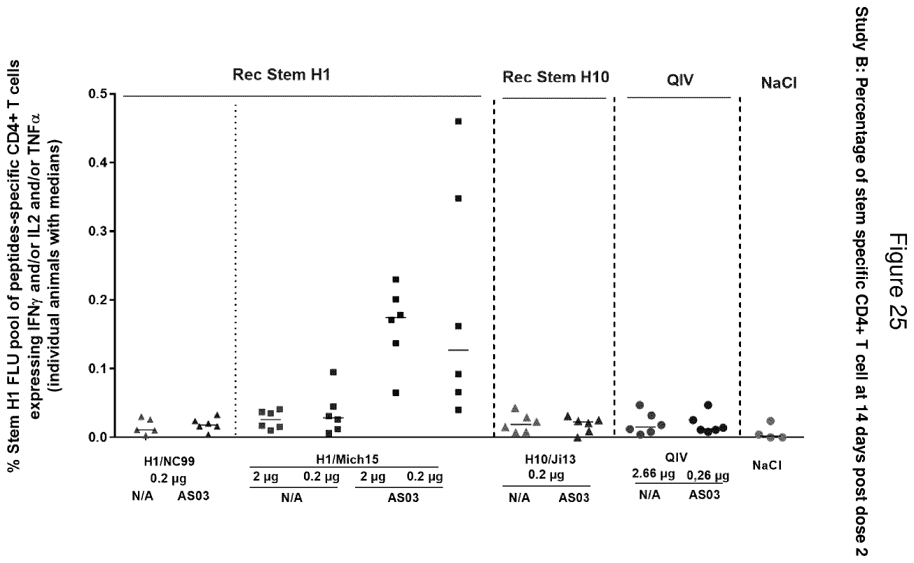

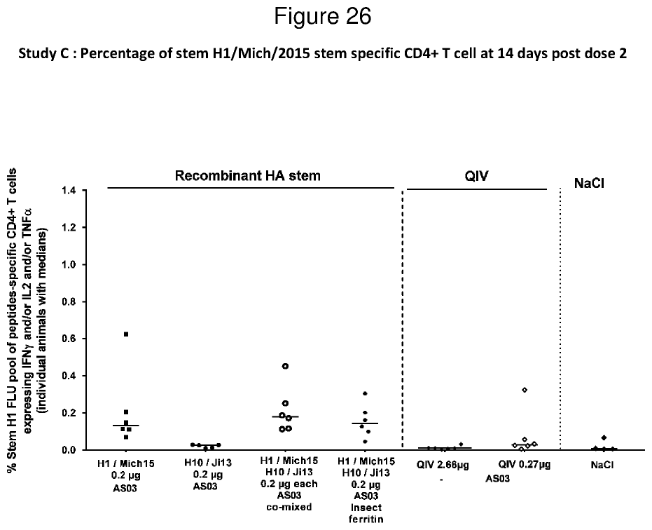

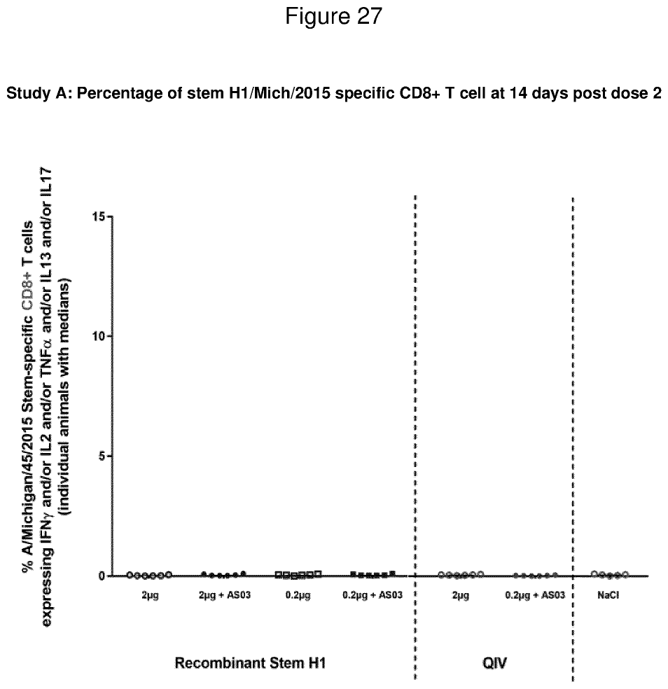

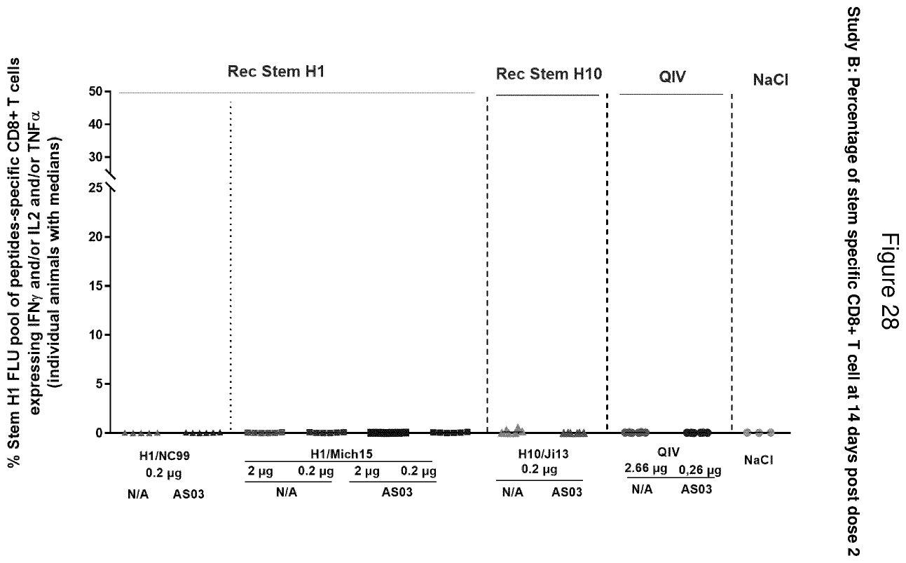

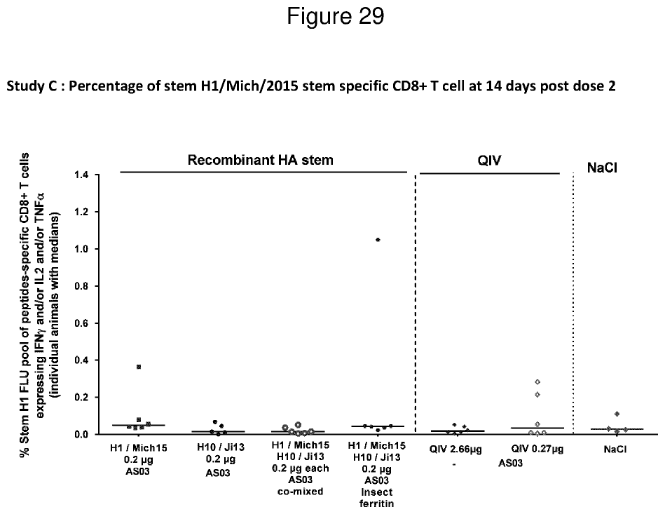

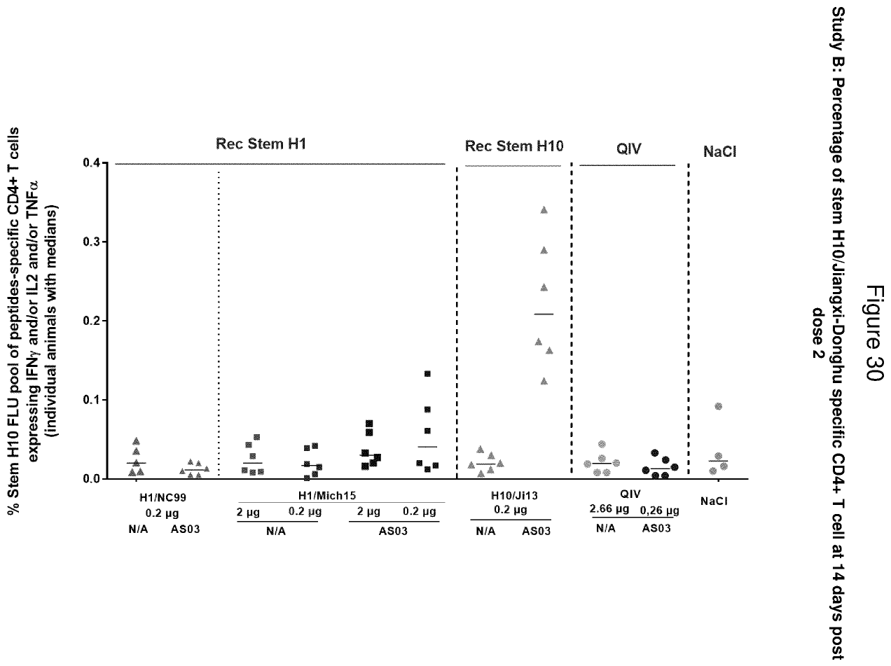

The T cell response induced by the stem H1 candidate vaccine was evaluated. The percentage of H1 stem-specific CD4+ T cells (Study A, FIG. 24 and Study B, FIG. 25 and Study C, FIG. 26) and CD8+ T cells (Study A, FIG. 27 and Study B, FIG. 28 and Study C, FIG. 29) were measured 14 days after the second immunization. Intracellular staining was performed on splenocytes after a 6 hours re-stimulation with peptide pools covering the sequence of H1 stem (A/Michigan/45/2015)).

For Study B, higher frequencies of H1/Mich/15 stem specific CD4+ T cell were induced by H1/Mich15 stem recombinant antigen (homotypic construct H. Pylori ferritin) adjuvanted with AS03 than the non-adjuvanted H1/Mich15 stem recombinant antigen.

For Study C, higher frequencies of H1/Mich/15 stem specific CD4+ T cell were observed with the H1/Mich15 homotypic construct, the recombinant H1/H10 stem heterotypic construct or the co-mixed formulation compared to QIV with or without AS03.

The dotted horizontal line on the figures corresponds to the threshold of detection.

### Example 8—H10/Jiangxi-Donghu Stem Specific CD4+ and CD8+ T Cells Frequencies at 14 Days Post Dose 2

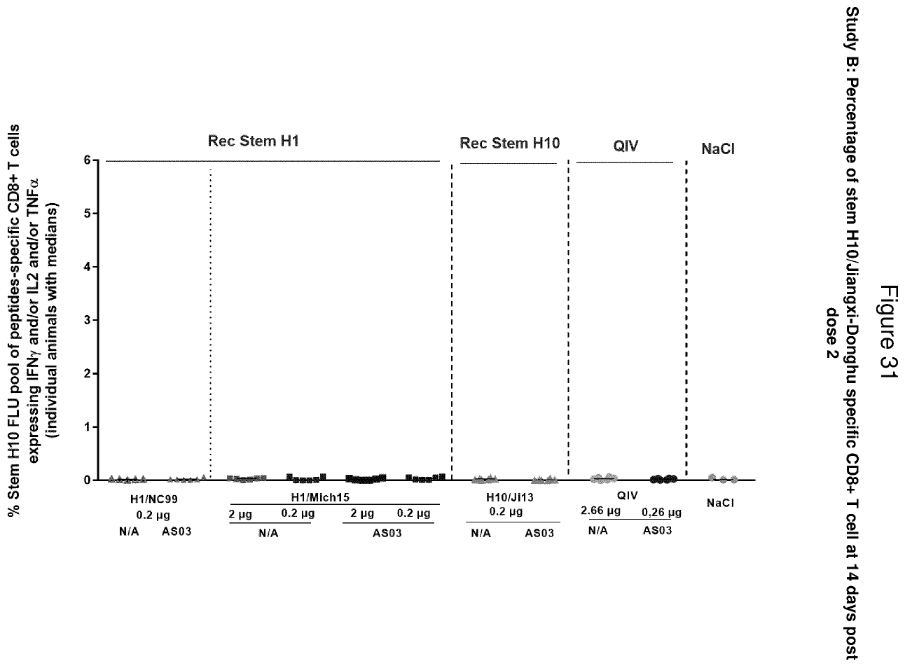

This experiment was carried out for Study B. The percentage of H10 stem-specific CD4+ T cells (FIG. 30) and CD8+ T cells (FIG. 31) were measured 14 days after the second immunization. Intracellular staining was performed on splenocytes after a 6 hours re-stimulation with peptide pools covering the sequence of H10 stem (H10/Jiangxi-Donghu)).

For Study B, higher frequencies of H10/Ji13 stem specific CD4+ T cell were induced by H10/Ji13 stem recombinant antigen (homotypic construct H. Pylori ferritin) adjuvanted with AS03 than the non-adjuvanted H1/Ji13 stem recombinant antigen. H10/Ji13 stem recombinant antigen adjuvanted with AS03 elicited higher H10/Ji13 stem specific CD4+ T cell response compared to QIV-AS03

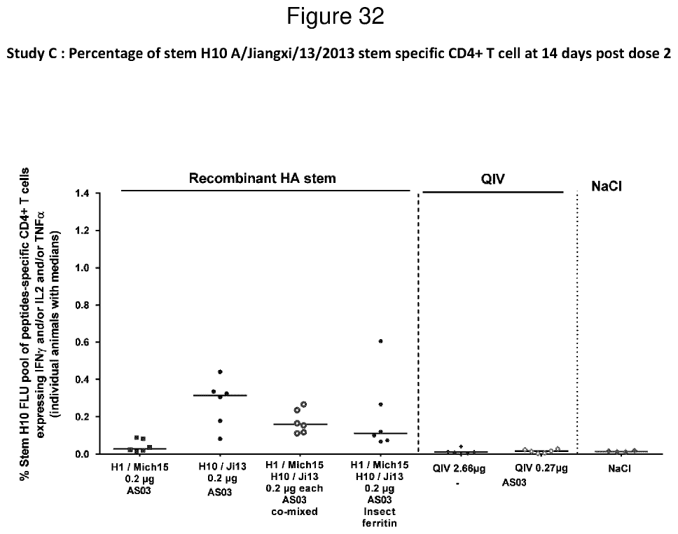

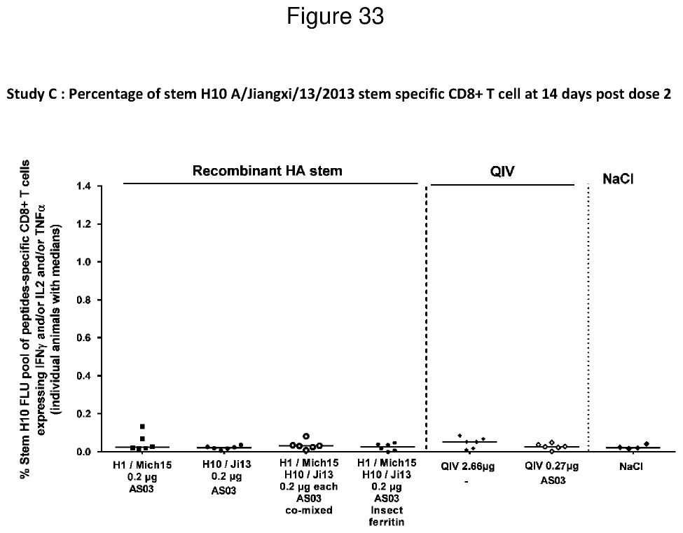

This experiment was further carried out for Study C. The percentage of H10 stem-specific CD4+ T cells (FIG. 32) and CD8+ T cells (FIG. 33) was measured 14 days after the second immunization. Intracellular staining was performed on splenocytes after a 6 hours restimulation with peptide pools covering the sequence of H10 stem (H10/Jiangxi-Donghu). Higher frequencies of H10/Jiangxi/13/2013 stem specific CD4+ T cell were observed with the H10/Ji13 homotypic construct, the Recombinant H1/H10 stem heterotypic construct or the co-mixed formulation compared to QIV with or without AS03.

The dotted horizontal line on the figures corresponds to the threshold of detection.

### Example 9—Group A1 H1/Mich/15, H1/NC/99 and H5/Vn/04 Microneutralization Titers at 14 Days Post Dose 2

In order to characterize the functional activity of the anti-H1 stem antibody response in vitro, neutralizing activity of the anti-H1 stem antibodies was evaluated by a neutralization assay.

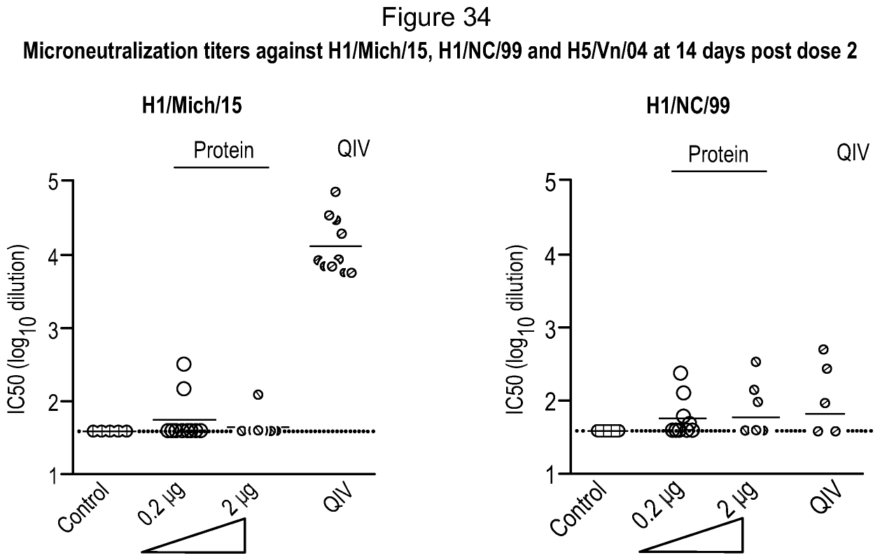

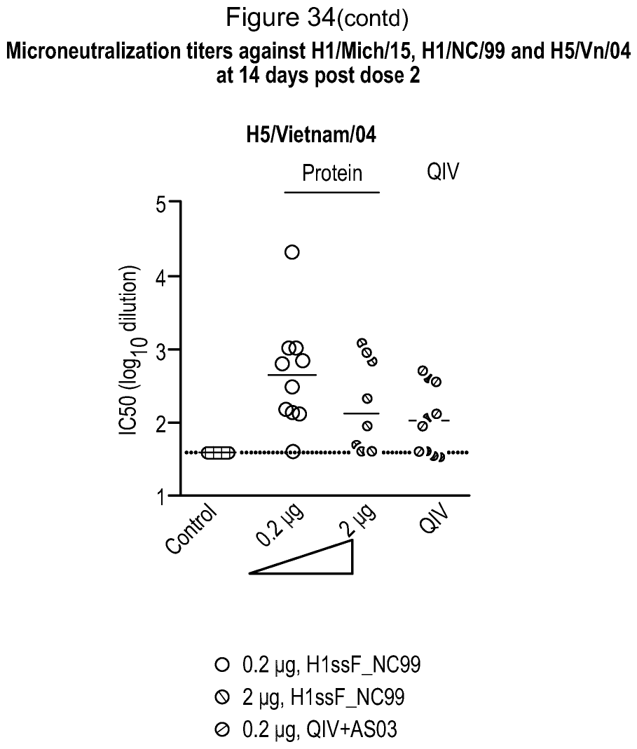

Microneutralization titers towards group A1 influenza virus were measured by microneutralisation assay using a H1/Mich/15, H1/NC/99 or H5/Vn/04 reporter viruses (FIG. 34). The results are expressed as IC50 (log₁₀ dilution).

The dotted horizontal line on the figures corresponds to the threshold of detection.

H1 stem NC99 recombinant antigen adjuvanted with AS03 elicited anti-H1/NC99 and anti-H5/VN04 neutralizing antibodies.

### REFERENCES

Carmona Martinez A, et al. A phase 1, open-label safety and immunogenicity study of an AS03-adjuvanted trivalent inactivated influenza vaccine in children aged 6 to 35 months. Hum Vaccin Immunother. 2014 10(7):1959-1968
Carter C, et al. A structure-function approach to optimizing TLR4 ligands for human vaccines, Clinical & Translation Immunology, 2016, 5, e108.
Cohet C, et al. Safety of AS03-adjuvanted Influenza Vaccines: A Review of the Evidence Vaccine 2019 37(23):3006-3021
Corbett, et al. Design of Nanoparticulate Group 2 Influenza Virus Hemagglutinin Stem Antigens That Activate Unmutated Ancestor B Cell Receptors of Broadly Neutralizing Antibody Lineages, mBio. 2019 10(1):e02810-18
Corti, et al. A neutralizing antibody selected from plasma cells that binds to group 1 and group 2 influenza A hemagglutinins, Science 2011 333, 850-856
Dayhoff M, O et al. A model of evolutionary change in proteins, Atlas of Protein Sequence and Structure, 1978 vol 5, supp. 3.
Gargon N, et al. Development and Evaluation of AS03, an Adjuvant System Containing α-Tocopherol and Squalene in an Oil-In-Water Emulsion Expert Rev Vaccines 2012 11:349-366
Fox C. Squalene Emulsions for Parenteral Vaccine and Drug Delivery Molecules 2009 14:3286-3312
Georgiev, I. S, et al. Two-Component Ferritin Nanoparticles for Multimerization of Diverse Trimeric Antigens ACS Infectious Diseases 2018 4(5): 788-796
Jackson L, et al. Effect of Varying Doses of a Monovalent H7N9 Influenza Vaccine With and Without AS03 and MF59 Adjuvants on Immune Response: A Randomized Clinical Trial JAMA 2015 314(3):237-246
Klucker M-F, et al. AF03, an Alternative Squalene Emulsion-Based Vaccine Adjuvant Prepared by a Phase Inversion Temperature Method J Pharm Sci. 2012 101(12):4490-4500
Lodaya R, et al. Formulation Design, Optimization and In Vivo Evaluations of an α-Tocopherol-Containing Self-Emulsified Adjuvant System using Inactivated Influenza Vaccine J Control Release 2019 316:12-21
Morel S, et al. Adjuvant System AS03 Containing α-Tocopherol Modulates Innate Immune Response and Leads to Improved Adaptive Immunity Vaccine 2011 29:2461-2473
Shah R, et al. The impact of size on particulate vaccine adjuvants. Nanomedicine (Lond) 2014 9:2671-2681
Shah R, et al. The development of self-emulsifying oil-in-water emulsion adjuvant and an evaluation of the impact of droplet size on performance. J Pharm Sci. 2015 104:1352-1361
Shah R, et al. The droplet size of emulsion adjuvants has significant impact on their potency, due to differences in immune cell-recruitment and -activation Scientific Reports 2019 9:11520
Steel J, et al. Influenza virus vaccine based on the conserved hemagglutinin stalk domain, mBio. 2010 1(1):e00018-10
Handbook of Pharmaceutical Excipients (eds. Rowe, Sheskey, & Quinn) 6th edition, 2009 ISBN 978-0-85369-792-3
Light Scattering from Polymer Solutions and Nanoparticle Dispersions Schartl W. 2007 ISBN 978-3-540-71950-2
WO2006/100109
WO2008/043774
WO2011/154444
WO2011/154443
WO2013/044203
WO2015/183969
WO2016172396
WO2018/045308
WO2018/005558
EP0868918

1. An immunogenic composition comprising: (i) at least one isolated influenza HA stem polypeptide, and (ii) a squalene emulsion adjuvant.

2. (canceled)

3. A method of eliciting an immune response in a subject, the method comprising administering to the subject (i) at least one isolated influenza HA stem polypeptide and (ii) a squalene emulsion adjuvant.

4. The immunogenic composition according to claim 1, wherein the isolated influenza HA stem polypeptide has been synthetically stabilised.

5. The immunogenic composition according to claim 1, wherein the isolated influenza HA stem polypeptide is presented in the form of a homotrimer.

6. The immunogenic composition according to claim 1, wherein the isolated influenza HA stem polypeptide is presented on a protein nanoparticle.

7. The immunogenic composition Or use or method according to claim 6, wherein the protein nanoparticle is ferritin.

8. The immunogenic composition according to claim 7, wherein the ferritin is selected from bacterial ferritin or insect ferritin.

9. The immunogenic composition according to claim 8, wherein the bacterial ferritin is H. pylori ferritin.

10. The immunogenic composition according to claim 8, wherein the insect ferritin is Trichoplusia ni ferritin.

11. The immunogenic composition according to claim 6, comprising a first isolated influenza HA stem polypeptide and a second isolated influenza HA stem polypeptide, where the first and second influenza HA stem polypeptides are derived from different influenza A strains, subtypes or groups.

12. The immunogenic composition according to claim 11, comprising one influenza HA stem polypeptide from an H1 strain and one influenza HA stem polypeptide from an H3 or an H10 strain.

13. The immunogenic composition according to claim 11, wherein the first and the second isolated HA stem polypeptides are presented on the same nanoparticle.

14. The immunogenic composition according to claim 13, wherein the nanoparticle comprises a fusion protein of a first isolated influenza HA stem polypeptide with insect ferritin heavy chain and a second isolated influenza stem polypeptide with insect ferritin light chain.

15. (canceled)

16. The immunogenic composition or use or method according to claim 6, wherein the linker comprises or consists of the polypeptide sequence SGG, or comprises or consists of the polypeptide sequence GGSGG [SEQ ID NO: 12].

17. The immunogenic composition according to claim 1, wherein the isolated influenza HA stem polypeptide comprises or consists of an amino acid sequence having at least 90% or at least 95% or at least 98% or at least 99% identity to the amino acid sequence set forth in any one of SEQ ID NO: 1, SEQ ID NO: 2, SEQ ID NO: 3 or SEQ ID NO: 4.

18. The immunogenic composition according to claim 17, wherein the isolated influenza HA stem polypeptide comprises or consists of the amino acid sequence set forth in any one of SEQ ID NO: 1, SEQ ID NO: 2, SEQ ID NO: 3 or SEQ ID NO: 4.

19. (canceled)

20. (canceled)

21. The immunogenic composition according to claim 1, wherein the squalene emulsion adjuvant comprises a surfactant and wherein the surfactant comprises or consists of polysorbate 80.

22. (canceled)

23. The immunogenic composition according to claim 21, wherein the amount of surfactant in a single dose of the squalene emulsion adjuvant is at least 0.4 mg, or 0.4 to 9.5 mg.

24. (canceled)

25. (canceled)

26. (canceled)

27. The immunogenic composition according to claim 1, wherein the squalene emulsion adjuvant consists essentially of squalene, polysorbate 80 and water.

28. (canceled)

29. The immunogenic composition according to claim 1, wherein the squalene emulsion adjuvant consists essentially of squalene, D/L-alpha-tocopherol, polysorbate 80 and water.

30. (canceled)

31. A method of administration of an immunogenic composition comprising (i) at least one isolated influenza HA stem polypeptide, and (ii) a squalene emulsion adjuvant, wherein administration of the isolated influenza HA stem polypeptide and the squalene emulsion adjuvant induces an immune response that is at least 2-fold, at least 5-fold, at least 10-fold, or at least 100-fold greater than that of the isolated influenza HA stem polypeptide administered without squalene emulsion adjuvant.

32. A kit comprising:
(i) a first container comprising at least one isolated influenza HA stem polypeptide according to claim 1; and
(ii) a second container comprising a squalene emulsion adjuvant according to claim 1.

33. (canceled)


SEQUENCE LISTING (16 sequences)

SEQ ID NO: 1 - protein, 198 aa, Influenza A virus
DTICIGYHANNSTDTVDTVLEKNVTVTHSVNLGSGLRMVTGLRNIPQRETRGLFGAIAGFIEGGWTGMVDGWYGYHHQNEQGSGYAADQKSTQNAINGITNMVNSVIEKMGSGGSGTDLAELLVLLLNERTLDFHDSNVKNLYEKVKSQLKNNAKEIGNGCFEFYHKCNNECMESVKNGTYDYPKYSEESKLNREKID

SEQ ID NO: 2 - protein, 198 aa, Influenza A virus
DTLCIGYHANNSTDTVDTVLEKNVTVTHSVNLGSGLRLATGLRNVPSIQSRGLFGAIAGFIEGGWTGMVDGWYGYHHQNEQGSGYAADLKSTQNAIDKITNMVNSVIEKMGSGGSGTYNAELLVLLLNERTLDYHDSNVKNLYEKVRNQLKNNAKEIGNGCFEFYHKCDNTCMESVKNGTYDYPKYSEEAKLNREKID

SEQ ID NO: 3 - protein, 212 aa, Influenza A virus
QKLPGNDNSTATLCLGHHAVPNGTIVKTITNDQIEVTNATELVFPGCGVLKLATGMRNVPEKQTRGIFGAIAGFIENGWEGMVDGWYGFRHQNSEGIGQAADLKSTQAAINQINGMVNRVIELMEQGGPDCYLAELLVALLNQHVIDLTDSEMRKLFERTKKQLRENAEDMGNGCFKIYHKCDNACIGSIRNGTYDHDVYRDEALNNRFQIK

SEQ ID NO: 4 - protein, 203 aa, Influenza A virus
DKICLGHHAVANGTIVKTLTNEQEEVTNATELVFPGCGVLMLATGMRNVPELIQGRGLFGAIAGFLENGWEGMVDGWYGFRHQNAQGTGQAADYKSTQAAIDQITGMVNRVVELMEQGGPDCYLAELLVAMLNQHVIDMADSEMRNLYERVRKQLRQNAEEDGKGCFEIYHACDDSCMESIRNNTYDHSQYREEALLNRLNIN

SEQ ID NO: 5 - protein, 163 aa, Helicobacter pylori
DIIKLLNEQVNKEMQSSNLYMSMSSWCYTHSLDGAGLFLFDHAAEEYEHAKKLIIFLNENNVPVQLTSISAPEHKFEGLTQIFQKAYEHEQHISESINNIVDHAIKSKDHATFNFLQWYVAEQHEEEVLFKDILDKIELIGNENHGLYLADQYVKGIAKSRKS

SEQ ID NO: 6 - protein, 383 aa, Artificial Sequence
  Polypeptide sequence of H1ssF_pylori (signal peptide-stabilised HA stem from A/New Caledonia/20/1999 (H1N1)-SGG-H. pylori ferritin)
MKAKLLVLLCTFTATYADTICIGYHANNSTDTVDTVLEKNVTVTHSVNLGSGLRMVTGLRNIPQRETRGLFGAIAGFIEGGWTGMVDGWYGYHHQNEQGSGYAADQKSTQNAINGITNMVNSVIEKMGSGGSGTDLAELLVLLLNERTLDFHDSNVKNLYEKVKSQLKNNAKEIGNGCFEFYHKCNNECMESVKNGTYDYPKYSEESKLNREKIDSGGDIIKLLNEQVNKEMQSSNLYMSMSSWCYTHSLDGAGLFLFDHAAEEYEHAKKLIIFLNENNVPVQLTSISAPEHKFEGLTQIFQKAYEHEQHISESINNIVDHAIKSKDHATFNFLQWYVAEQHEEEVLFKDILDKIELIGNENHGLYLADQYVKGIAKSRKSGS

SEQ ID NO: 7 - protein, 381 aa, Artificial Sequence
  Polypeptide sequence of H1ssF_pylori (signal peptide-stabilised HA stem from A/Michigan/45/2015 (H1N1)-SGG-H. pylori ferritin)
MKAILVVLLYTFTTANADTLCIGYHANNSTDTVDTVLEKNVTVTHSVNLGSGLRLATGLRNVPSIQSRGLFGAIAGFIEGGWTGMVDGWYGYHHQNEQGSGYAADLKSTQNAIDKITNMVNSVIEKMGSGGSGTYNAELLVLLLNERTLDYHDSNVKNLYEKVRNQLKNNAKEIGNGCFEFYHKCDNTCMESVKNGTYDYPKYSEEAKLNREKIDSGGDIIKLLNEQVNKEMQSSNLYMSMSSWCYTHSLDGAGLFLFDHAAEEYEHAKKLIIFLNENNVPVQLTSISAPEHKFEGLTQIFQKAYEHEQHISESINNIVDHAIKSKDHATFNFLQWYVAEQHEEEVLFKDILDKIELIGNENHGLYLADQYVKGIAKSRKS

SEQ ID NO: 8 - protein, 399 aa, Artificial Sequence
  Polypeptide sequence of H3ssF_pylori (signal peptide-stabilised HA stem from A/Finland/486/2004 (H3N2)-SGG-H. pylori ferritin)
MKTIIALSYILCLVFAQKLPGNDNSTATLCLGHHAVPNGTIVKTITNDQIEVTNATELVFPGCGVLKLATGMRNVPEKQTRGIFGAIAGFIENGWEGMVDGWYGFRHQNSEGIGQAADLKSTQAAINQINGMVNRVIELMEQGGPDCYLAELLVALLNQHVIDLTDSEMRKLFERTKKQLRENAEDMGNGCFKIYHKCDNACIGSIRNGTYDHDVYRDEALNNRFQIKSGGDIIKLLNEQVNKEMQSSNLYMSMSSWCYTHSLDGAGLFLFDHAAEEYEHAKKLIIFLNENNVPVQLTSISAPEHKFEGLTQIFQKAYEHEQHISESINNIVDHAIKSKDHATFNFLQWYVAEQHEEEVLFKDILDKIELIGNENHGLYLADQYVKGIAKSRKSGSRKS

SEQ ID NO: 9 - protein, 386 aa, Artificial Sequence
  Polypeptide sequence of H10ssF_pylori (signal peptide-stabilised HA stem from A/Jiangxi/IPB13/2013 (H10N8)-SGG-H. pylori ferritin)
MYKIVVIIALLGAVKGLDKICLGHHAVANGTIVKTLTNEQEEVTNATELVFPGCGVLMLATGMRNVPELIQGRGLFGAIAGFLENGWEGMVDGWYGFRHQNAQGTGQAADYKSTQAAIDQITGMVNRVVELMEQGGPDCYLAELLVAMLNQHVIDMADSEMRNLYERVRKQLRQNAEEDGKGCFEIYHACDDSCMESIRNNTYDHSQYREEALLNRLNINSGGDIIKLLNEQVNKEMQSSNLYMSMSSWCYTHSLDGAGLFLFDHAAEEYEHAKKLIIFLNENNVPVQLTSISAPEHKFEGLTQIFQKAYEHEQHISESINNIVDHAIKSKDHATFNFLQWYVAEQHEEEVLFKDILDKIELIGNENHGLYLADQYVKGIAKSRKS

SEQ ID NO: 10 - protein, 173 aa, Trichoplusia ni
RSCRNSMRQQIQMEVGASLQYLAMGAHFSKDVVNRPGFAQLFFDAASEEREHAMKLIEYLLMRGELTNDVSSLLQVRPPTRSSWKGGVEALEHALSMESDVTKSIRNVIKACEDDSEFNDYHLVDYLTGDFLEEQYKGQRDLAGKASTLKKLMDRHEALGEFIFDKKLLGIDV

SEQ ID NO: 11 - protein, 183 aa, Trichoplusia ni
EYGSHGNVATELQAYAKLHLERSYDYLLSAAYFNNYQTNRAGFSKLFKKLSDEAWSKTIDIIKHVTKRGDKMNFDQHSTMKTERKNYTAENHELEALAKALDTQKELAERAFYIHREATRNSQHLHDPEIAQYLEEEFIEDHAEKIRTLAGHTSDLKKFITANNGHDLSLALYVFDEYLQKTV

SEQ ID NO: 12 - protein, 5 aa, Artificial Sequence
  Linker sequence
GGSGG

SEQ ID NO: 13 - protein, 450 aa, Artificial Sequence
  Polypeptide sequence of insect ferritin construct iFH-F2A-iFL-6R (single polypeptide; insect ferritin heavy chain-self-cleaving sequence-insect ferritin light chain)
MDSKGSSQKGSRLLLLLVVSNLLLPQGVVARSCRNSMRQQIQMEVGASLQYLAMGAHFSKDVVNRPGFAQLFFDAASEEREHAMKLIEYLLMRGELTNDVSSLLQVRPPTRSSWKGGVEALEHALSMESDVTKSIRNVIKACEDDSEFNDYHLVDYLTGDFLEEQYKGQRDLAGKASTLKKLMDRHEALGEFIFDKKLLGIDVRRRRRRSGSGAPVKQTLNFDLLKLAGDVESNPGPMDSKGSSQKGSRLLLLLVVSNLLLPQGVVAEYGSHGNVATELQAYAKLHLERSYDYLLSAAYFNNYQTNRAGFSKLFKKLSDEAWSKTIDIIKHVTKRGDKMNFDQHSTMKTERKNYTAENHELEALAKALDTQKELAERAFYIHREATRNSQHLHDPEIAQYLEEEFIEDHAEKIRTLAGHTSDLKKFITANNGHDLSLALYVFDEYLQKTV

SEQ ID NO: 14 - protein, 843 aa, Artificial Sequence
  Polypeptide sequence of H1Mich15ss_iH-F2A-H3ss_iL-6R (H1/H3 ssF insect - single polypeptide, self-cleaving)
MKAILVVLLYTFTTANADTLCIGYHANNSTDTVDTVLEKNVTVTHSVNLGSGLRLATGLRNVPSIQSRGLFGAIAGFIEGGWTGMVDGWYGYHHQNEQGSGYAADLKSTQNAIDKITNMVNSVIEKMGSGGSGTYNAELLVLLLNERTLDYHDSNVKNLYEKVRNQLKNNAKEIGNGCFEFYHKCDNTCMESVKNGTYDYPKYSEEAKLNREKIDGGSGGRSCRNSMRQQIQMEVGASLQYLAMGAHFSKDVVNRPGFAQLFFDAASEEREHAMKLIEYLLMRGELTNDVSSLLQVRPPTRSSWKGGVEALEHALSMESDVTKSIRNVIKACEDDSEFNDYHLVDYLTGDFLEEQYKGQRDLAGKASTLKKLMDRHEALGEFIFDKKLLGIDVRRRRRRSGSGAPVKQTLNFDLLKLAGDVESNPGPMKTIIALSYILCLVFAQKLPGNDNSTATLCLGHHAVPNGTIVKTITNDQIEVTNATELVFPGCGVLKLATGMRNVPEKQTRGIFGAIAGFIENGWEGMVDGWYGFRHQNSEGIGQAADLKSTQAAINQINGMVNRVIELMEQGGPDCYLAELLVALLNQHVIDLTDSEMRKLFERTKKQLRENAEDMGNGCFKIYHKCDNACIGSIRNGTYDHDVYRDEALNNRFQIKGGSGGEYGSHGNVATELQAYAKLHLERSYDYLLSAAYFNNYQTNRAGFSKLFKKLSDEAWSKTIDIIKHVTKRGDKMNFDQHSTMKTERKNYTAENHELEALAKALDTQKELAERAFYIHREATRNSQHLHDPEIAQYLEEEFIEDHAEKIRTLAGHTSDLKKFITANNGHDLSLALYVFDEYLQKTV

SEQ ID NO: 15 - protein, 835 aa, Artificial Sequence
  Polypeptide sequence of H1Mich15ss_iH-F2A-H10ss_iL-6R (H1/H10 ssF insect - single polypeptide, self-cleaving)
MKAILVVLLYTFTTANADTLCIGYHANNSTDTVDTVLEKNVTVTHSVNLGSGLRLATGLRNVPSIQSRGLFGAIAGFIEGGWTGMVDGWYGYHHQNEQGSGYAADLKSTQNAIDKITNMVNSVIEKMGSGGSGTYNAELLVLLLNERTLDYHDSNVKNLYEKVRNQLKNNAKEIGNGCFEFYHKCDNTCMESVKNGTYDYPKYSEEAKLNREKIDGGSGGRSCRNSMRQQIQMEVGASLQYLAMGAHFSKDVVNRPGFAQLFFDAASEEREHAMKLIEYLLMRGELTNDVSSLLQVRPPTRSSWKGGVEALEHALSMESDVTKSIRNVIKACEDDSEFNDYHLVDYLTGDFLEEQYKGQRDLAGKASTLKKLMDRHEALGEFIFDKKLLGIDVRRRRRRSGSGAPVKQTLNFDLLKLAGDVESNPGPMYKIVVIIALLGAVKGLDKICLGHHAVANGTIVKTLTNEQEEVTNATELVFPGCGVLMLATGMRNVPELIQGRGLFGAIAGFLENGWEGMVDGWYGFRHQNAQGTGQAADYKSTQAAIDQITGMVNRVVELMEQGGPDCYLAELLVAMLNQHVIDMADSEMRNLYERVRKQLRQNAEEDGKGCFEIYHACDDSCMESIRNNTYDHSQYREEALLNRLNINGGSGGEYGSHGNVATELQAYAKLHLERSYDYLLSAAYFNNYQTNRAGFSKLFKKLSDEAWSKTIDIIKHVTKRGDKMNFDQHSTMKTERKNYTAENHELEALAKALDTQKELAERAFYIHREATRNSQHLHDPEIAQYLEEEFIEDHAEKIRTLAGHTSDLKKFITANNGHDLSLALYVFDEYLQKTV

SEQ ID NO: 16 - protein, 835 aa, Artificial Sequence
  Polypeptide sequence of H1NC99ss_iH-F2A-H10ss_iL-6R (H1/H10 ssF insect - single polypeptide, self-cleaving)
MKAKLLVLLCTFTATYADTICIGYHANNSTDTVDTVLEKNVTVTHSVNLGSGLRMVTGLRNIPQRETRGLFGAIAGFIEGGWTGMVDGWYGYHHQNEQGSGYAADQKSTQNAINGITNMVNSVIEKMGSGGSGTDLAELLVLLLNERTLDFHDSNVKNLYEKVKSQLKNNAKEIGNGCFEFYHKCNNECMESVKNGTYDYPKYSEESKLNREKIDGGSGGRSCRNSMRQQIQMEVGASLQYLAMGAHFSKDVVNRPGFAQLFFDAASEEREHAMKLIEYLLMRGELTNDVSSLLQVRPPTRSSWKGGVEALEHALSMESDVTKSIRNVIKACEDDSEFNDYHLVDYLTGDFLEEQYKGQRDLAGKASTLKKLMDRHEALGEFIFDKKLLGIDVRRRRRRSGSGAPVKQTLNFDLLKLAGDVESNPGPMYKIVVIIALLGAVKGLDKICLGHHAVANGTIVKTLTNEQEEVTNATELVFPGCGVLMLATGMRNVPELIQGRGLFGAIAGFLENGWEGMVDGWYGFRHQNAQGTGQAADYKSTQAAIDQITGMVNRVVELMEQGGPDCYLAELLVAMLNQHVIDMADSEMRNLYERVRKQLRQNAEEDGKGCFEIYHACDDSCMESIRNNTYDHSQYREEALLNRLNINGGSGGEYGSHGNVATELQAYAKLHLERSYDYLLSAAYFNNYQTNRAGFSKLFKKLSDEAWSKTIDIIKHVTKRGDKMNFDQHSTMKTERKNYTAENHELEALAKALDTQKELAERAFYIHREATRNSQHLHDPEIAQYLEEEFIEDHAEKIRTLAGHTSDLKKFITANNGHDLSLALYVFDEYLQKTV